(19) United States
(12) Patent Application Publication
(10) Pub. No.: US 2008/0199438 A1
(43) Pub. Date: Aug. 21, 2008

(54) Methods For Suppressing Tumor Proliferation

(75) Inventors: Katsuo Sueishi, Fukuoka (JP); Yoshikazu Yonemitsu, Chiba (JP); Yasunori Shikada, Oita (JP); Norifumi Tsutsumi, Ehime (JP); Mamoru Hasegawa, Ibaraki (JP)

Correspondence Address: CLARK & ELBING LLP, 101 FEDERAL STREET, BOSTON, MA, 02110, US

(73) Assignee: DNAVEC RESEARCH INC., IBARAKI (JP)

(21) Appl. No.: 10/598,947

(22) PCT Filed: Mar. 15, 2005
(86) PCT No.: PCT/JP2005/004485

§ 371 Date: Mar. 27, 2007

(30) Foreign Application Priority Data
Mar. 16, 2004 (JP) 2004-074570

(51) Int. Cl. A61K 48/00; A61K 31/7105; A61K 38/02; A61P 35/00

(52) U.S. Cl. 424/93.7; 424/93.2; 514/44; 514/2

(57) ABSTRACT

The present invention provides methods for suppressing tumor proliferation comprising the step of inhibiting the expression of PDGF-A or the binding between PDGF-A homodimers and PDGFRα. Activation of the PDGFRα-p70S6K signal transduction pathway by PDGF-AA is an important factor in tumor angiogenesis and relates to the prognosis of patients suffering from tumors. By inhibiting PDGF-A expression in tumors or in their surrounding tissues, or by inhibiting the binding between PDGF-A homodimers and PDGFRα, the formation and retention of tumor vasculature can be inhibited, thereby suppressing tumor proliferation.

### TECHNICAL FIELD

The present invention relates to methods for suppressing tumor proliferation.

### BACKGROUND ART

Many animal experiments have shown reduced tumor proliferation due to anti-angiogenesis drugs, showing that angiogenesis is necessary for tumor expansion (Folkman J., N Engl J Med 285: 1182-1186 (1971); Holmgren L. et al., Nat. Med. 1: 149-153 (1995); Hlatky L et al, J Natl Cancer Inst. 94: 883-893 (2002)). Vascular endothelial growth factor (VEGF) is a key mediator of tumor angiogenesis, and inhibition of VEGF activity by overexpression of fms-like tyrosine kinase-1 (FLT-1), a soluble high-afflinity receptor for VEGF, induces tumor dormancy (Goldman C K et al., Proc Natl Acad Sci USA 95: 8795-8800 (1988); Kio C J et al., Proc Natl Acad Sci USA 98: 4605-4610 (2001)). These studies suggest that signal transduction involving VEGE could be a target for tumor angiogenesis. However, another study reported that FLT-1's anti-tumor effect was highly dependant on the VEGF expression level in each of the tumor types examined (Takayama K et al., Cancer Res 60: 2169-2177 (2000)), suggesting that therapeutic strategies using anti-VEGF effects are quite limited. Thus, to develop broad-spectrum anti-tumor drugs, common molecular targets for tumor angiogenesis, which do not depend on the expression profile of angiogenic growth factors in each tumor type, were required.

[Non-Patent Document 1] Folkman J., N Engl J Med 285: 1182-1186 (1971)

[Non-Patent Document 2] Holmgren L. et al., Nat. Med. 1: 149-153 (1995)

[Non-Patent Document 3] Hlatky L et al., J Natl Cancer Inst. 94: 883-893 (2002)

[Non-Patent Document 4] Goldman C K et al., Proc Natl Acad Sci, USA. 95: 8795-8800 (1988)

[Non-Patent Document 5] Kuo C J et al., Proc Natl Acad Sci USA. 98: 4605-4610 (2001)

[Non-Patent Document 6] Takayama K et al., Cancer Res. 60: 2169-2177 (2000)

### DISCLOSURE OF THE INVENTION

The present invention provides methods for suppressing tumor proliferation by inhibiting the formation and retention of tumor vasculature.

Rapamycin (RAPA), a new immunosuppressive drug developed in recent studies, has anti-angiogenic activity and has been shown to shrink tumors (Guba M et al., Nat. Med. 8: 128-135 (2002)). Immunosuppressive therapy after organ transplant increases the risks of tumor generation and regeneration in patients, whereas use of RAPA is considered to reduce the chance of malignant tumor generation. Data from cultured cells suggests that RAPA's anti-angiogenic effect involves a reduction in VEGF expression in tumors, but the precise mode of action in vivo is unclear.

Separately from this, the present inventors recently proved that expression of a polypeptide involved in angiogenesis in mesenchymal cells (MCs), but not in endothelial cells (ECs), plays an essential role in therapeutic angiogenesis for the therapy of severe limb ischemia using fibroblast growth factor-2 (FGF-2) (Masaki I et al., Circ Res. 90: 966-973 (2002); Onimaru M et al., Circ Res. 91: 723-730 (2002)). FGF-2 stimulates local expression of VEGF and another angiogenic growth factor, hepatocyte growth factor/scatter factor (HGF/SF), in vascular mesenchymal cells (MCs: including pericytes, vascular smooth muscle cells, and adventitial fibroblasts) (Onimaru M et al., Circ Res. 91: 723-730 (2002)). Interestingly, time courses of FGF-2-mediated HGF/SF expression are biphasic, meaning that upregulation in the early phase does not require new protein synthesis, but that upregulation in the late phase is mediated and sustained by the endogenous platelet-derived growth factor receptor-α (PDGFRα)-p70S6 kinase pathway (Onimaru M et al., Circ Res. 91: 723-730 (2002)).

The present inventors hypothesized that in host-derived stromal MCs the PDGFRα-p70S6K signal transduction pathway is involved in RAPAs antitumor effect regardless of the various angiogenic signals from each tumor, since not only VEGF but also host-derived FGF-2 activities are expected to be involved in tumor expansion (Compagni A et al., Cancer Res. 60: 7163-7169 (2000)), and also since RAPA is a specific inhibitor of p70S6K via lowering of TOR (target of rapamycin) activity.

In fact, by using tumor-free assay systems (i.e. mouse limb ischemia), the present inventors proved that p70S6K inhibitor rapamycin (RAPA) uses MCs as a target, silencing the PDGFRα-p70S6K pathway and thus blocking the continuous expression of vascular endothelial growth factor (VEGF) and hepatocyte growth factor (HGF) (Example 2). In addition, in assessments using tumors, RAPA invariably induced tumor dormancy and over time resulted in serious ischemic conditions, regardless of the variety of angiogenic factor expression profiles in each of the examined tumors, and even when VEGF expression in the tumors was enhanced (Example 4). Since RAPA displayed only a minimal influence on hypoxia-related VEGF expression in culture systems, these results suggested that in vivo RAPA targets the host-vasculature rather than the tumor itself. Namely, the present invention revealed that the PDEFRα-p70S6K pathway is an essential regulatory factor not only for FGF-2-mediated therapeutic angiogenesis, but also for host-derived vasculature in tumor angiogenesis, and also revealed that the PDEFRα-p70S6K pathway regulates expression of multiple angiogenic growth factors. Thus, the present invention proved that in MCs the PDGFRα-p70S6K signal transduction pathway is a common and ubiquitous molecular target that can inhibit angiogenesis regardless of the properties of malignant tumors.

The biological role of PDGFRα has long been the subject of argument, PDGF-A homodimers (PDCF-AA) induce the DNA synthesis and proliferation of NIH3T3 cells. On the other hand, however, in other cells they inhibit chemotaxis reactions induced by other reagents (Siegbahn A et al., J Clin Invest. 85: 916-920 (1990)). While there is little evidence of PDGF receptor expression in endothelial cells, PDGF receptor ligands, including not only PDGF-AA and PDGF-BB but also the novel PDGF, PDGF-CC (Li X et al., Nat Cell Biol. 2: 302-309 (2000), stimulate angiogenesis in vivo (Nicosia R F et al., Am J Pathol. 145: 1023-1029 (1994); Cao R et al., FASEB J. 16: 1575-1583 (2002)). These findings suggest the possibility that other angiogenesis-stimulating factors also mediate the PDGF-dependent angiogenesis process. In line with previous studies (Onimaru M et al., Circ Res. 91: 723-730 (2002)), the present invention suggests that the PDGFRα system is essential for sustaining the angiogenesis signals that use VEGF and HGF/SF in MCs. However, since all of these ligands activate PDGFRα and each can cause different cellular responses, the essential ligands for angiogenesis have not been determined. The present invention shows that of the PDGFRα ligands, PDGF-A in particular plays an important role in the formation of tumor vasculature. Since enhanced PDGF-A expression closely relates to tumor malignancy, tumor proliferation was dramatically suppressed upon inhibiting PDGF-A expression in tumor cells (Example 5). Thus, the present invention clarifies that inhibition of PDGF-A expression or inhibition of the binding between PDGF-AA and PDGFRα can result in efficient suppression of tumor angiogenesis, thereby bringing about tumor dormancy.

For example, it is possible to inhibit formation and retention of host-vasculature in tumors, to suppress tumor proliferation, and to further bring about tumor ischemia and tumor degeneration, by administering tumors with siRNAs that inhibit PDGF-A expression or vectors that express these siRNAs, or by administering tumors with soluble PDGFRα or anti-PDGF-A antibodies, or vectors that express either of these. These treatments enable specific inhibition of PDGFRα-p70S6 kinase signal transduction in the tumor vasculature, and show excellent therapeutic effects with few side effects. The methods of the present invention are extremely useful as novel anti-tumor therapeutic methods that can very efficiently induce tumor dormancy.

Accordingly, the present invention relates to methods for suppressing tumor proliferation, more specifically, it relates to the inventions set forth in each of claims. In addition, inventions comprising one or a combination of multiple inventions set forth in the claims citing the same claim are already included in the inventions set forth in these claims.

Specifically, the present invention relates to:

[1] a method for suppressing tumor proliferation, comprising the step of inhibiting the expression of a PDGF-A or the binding between a PDGF-A homodimer and a PDGFRα;

[2] the method of [1], wherein the step administers to a tumor a minus strand RNA virus vector encoding a secretory protein that binds to a PDGF-A homodimer or a PDGFRα;

[3] the method of [2], wherein a cell to which the vector has been introduced is administered;

[4] the method of [3], wherein the cell is a dendritic cell;

[5] the method of any one of [2] to [4], wherein the secretory protein is a soluble PDGFRα;

[6] the method of any one of [2] to [5], wherein the minus strand RNA virus vector is a Sendai virus vector;

[7] the method of [1], wherein the step administers to a tumor an antisense RNA or siRNA of a PDGF-A gene, or a vector encoding the antisense RNA or siRNA;

[8] the method of any one of [1] to [7], wherein the tumor is selected from the group consisting of a squamous cell carcinoma, a hepatocarcinoma, and an adenocarcinoma;

[9] an antitumor agent comprising a compound that inhibits the expression of a PDGF-A or the binding between a PDGF-A homodimer and a PDGFRα as an active ingredient;

[10] the antitumor agent of [9], wherein the agent comprises any one of (a) to (d) below:

(a) a secretory protein that binds to a PDGF-A homodimer or a PDGFRα,

(b) an antisense RNA of a PDGF-A gene or a PDGFRαgene,

(c) an siRNA of a PDGF-A gene or a PDGFRα gene, and

(d) a vector encoding any one of (a) to (c);

[11] the antitumor agent of [10], wherein the agent comprises a minus strand RNA virus vector encoding a secretory protein that binds to a PDGF-A homodimer or a PDGFRα;

[12] the antitumor agent of [10] or [11], wherein the secretory protein is a soluble PDGFRα;

[13] the antitumor agent of [11], wherein the minus strand RNA virus vector is a Sendai virus vector;

[14] the antitumor agent of any one of [10] to [13], wherein the agent comprises a cell, to which has been introduced a vector that encodes a secretory protein that binds to a PDGF-A homodimer or a PDGFRα;

[15] the antitumor agent of [14], wherein the cell is a dendritic cell;

[16] the antitumor agent of [10], wherein the agent comprises an antisense RNA or siRNA of a PDGF-A gene, or a vector encoding the antisense RNA or siRNA, as an active ingredient; and

[17] the antitumor agent of any one of [9] to [16], wherein the tumor is selected from the group consisting of a squamous cell carcinoma, a hepatocarcinoma, and an adenocarcinoma.

### BRIEF DESCRIPTION OF THE DRAWINGS

FIG. 1 shows analytical results for the mode of action of FGF-2 and PDGF-AA in the upregulation of VEGF expression.

(A) Recombinant FGF-2 and PDGF-AA work together to increase VEGF secretion from fibroblasts (MRC5) and vascular smooth muscle cells (HSMCs). After 48 hours of preincubation in serum-free conditions, each of the cell lines was stimulated with FGF-2 and/or PDGF-AA. After 72 hours, the cultured medium was subjected to ELISA. n3 in each group. *P<0.01. #P<0.05

(B) A time course of FGF-2-mediated expression of PDGFRα mRNA in MRC5 cells and HSMCs was analyzed by Northern blotting. After 48 hours of preincubation under serum-free conditions, each of the cell lines was stimulated with FGF-2. Cells were harvested at the time indicated in the figure and then subjected to Northern blot analysis. Bands were visualized and subjected to densitometric analysis using a photoimager. The experience was carried out in duplicate and similar results were obtained.

FIG. 2 shows that PDGFRα-p70S6K is essential for the sustained/biphasic FGF-2-mediated expression of VEGF/HGF in MCs.

(A) Effect of various inhibitors of intracellular signal transduction pathways upon the secretion of VEGF and HGF in MRC5 cells. After 48 hours of preincubation in the presence of 1% FBS, cells were stimulated with 10 ng/ml of human recombinant FGF-2 in the presence or absence of various inhibitors. After 72 hours, the medium was subjected to ELISA. n=3 in each group. *P<0.01.

(B) A p70S6K inhibitor, Rapamycin (RAPA) stops the later phase of FGF-2-mediated VEGF mRNA expression in MRC5 cells. After 48 hours of preincubation in the presence of 1% FBS, cells were stimulated with 10 ng/ml of recombinant human FGF-2. Cells were harvested at the time indicated in the figure and then subjected to Northern blot analysis. The bands were visualized and subjected to densitometric analysis using a photoimager. The graph shows the quantitative results of relative levels of VEGF mRNA, reflecting the results of triplicate experiments. *P<0.01.

(C) Increases in FGF-2-mediated VEGF secretion completely depend on PDGFRα. After 48 hours of preincubation in the presence of 1% FBS, MRC5 cells were stimulated with 10 ng/ml of recombinant human FGF-2 in the presence or absence of an anti-PDGFRα neutralizing antibody. After 72 hours, the medium was subjected to ELISA. Similar results were obtained for the expression of HGF (data not shown). *P<0.01.

FIG. 3 shows that upregulation of VEGF and HGF mediated by the PDGFRα system is essential for the therapeutic effect of FGF-2 gene transfer in mouse severe limb ischemia. *P<0.01. #P<0.05.

(A and B) Time courses of the relative expressions of PDGF-A (upper panel) and PDGFRα (lower panel) mRNAs in an ischemic femoral muscle of a C57BL6 limb salvage mouse model, with or without FGF-2 gene transfer SeV-mGF2 (10⁷ plaque forming units: pfu) was intramuscularly injected immediately after the limb ischemia-inducing surgery. Femoral muscle samples were prepared at each time and subjected to real-time PCR. Data were standardized using each GAPDH mRNA level and expression levels are shown relative to the results obtained with untreated control mice. Each group contains four mice. At each time, one or two ischemic mice injected with a control viral vector (SeV-luciferase) were used as control mice, and these mice showed results similar to those of the ischemic limb mice (data not shown).

(C and D) Time courses of the relative expressions of VEGF (upper panel) and HGF (lower panel) mRNAs in an ischemic femoral muscle of a C57BL6 limb salvage mouse model treated with an anti-PDGF-AA neutralizing antibody (refer to the FIG. 4 legend for the protocol) or RAPA (intraperitoneally injected everyday at 1.5 mg/kg/day), following FGF-2 gene transfer. Tissue samples the same as those of the ischemia and ischemia+FGF-2 groups of FIG. 3A were used. At each time, one or two ischemic mice injected with a control viral vector (SeV-luciferase) were used as control mice, and these mice showed results similar to those of the mice with ischemia alone (data not shown).

(E and F) RAPA inhibits FGF-2-mediated expression of VEGF (panel E) and HGF (panel F) proteins in the ischemic limb salvage mouse model. Intraperitoneal injection of RAPA (1.5 mg/kg/day, everyday) was initiated one day before day 0, and then the ischemia operation was carried out. At that time, 107 pfu of a control virus (SeV-luciferase) or SeV-mFGF2 was injected intramuscularly. Two days later, femoral muscle was subjected to ELISA. No difference was observed between the RAPA-treated and untreated mice in the exogenous expression of FGF-2 induced by FGF-2 gene transfer (data not shown).

FIG. 4 shows that the anti-PDGF-AA neutralizing antibody eliminates the effect of FGF-2 gene transfer in balb/c nu/nu mice exhibiting limb ischemia (limb autoamputation model), as is the case with RAPA. Limb prognosis was determined by 12 limb salvage scores and data were analyzed using log-rank tests. The anti-PDGF-AA neutralizing antibody was administered by continuous release (200 μg/7 days) into the peritoneal cavity via an implanted disposable osmotic pump. Immediately after the surgical induction of ischemia, an additional intraperitoneal bolus injection (100 μg) was also carried out.

FIG. 5 shows the effect of RAPA treatment and soluble PDGFRα expression on tumor proliferation. Each type of tumor cell was subcutaneously implanted at a dose of 16 cells, and after seven days RAPA (15 mg/kg/day) or 0.1 mol/L of phosphate buffered saline (PBS) was intraperitoneally injected every day, or SeV-luciferase or SeV-hsPDGFRα (1×10⁸ pfu/tumor) was injected into the tumors once. *P<0.01. P<0.05.

(A to D) In vitro expression profiles of angiogenic growth factors including PDGF-AA in SAS (human oral cavity-derived oral squamous cell carcinoma) and MH134 (mouse hepatoma), and tumor-inhibitory effect of RAPA. The data includes the results of three independent experiments where two to four mice were used in each experiment. On Day 28, an overall image was photographed. Arrows indicate tumors.

(E and F) Antitumor effect on SAS and MH134 of a recombinant SeV that expresses the extracellular domain of human PDGFRα. Five days after cell implantation, 50 μL of the vector solution was injected into the tumors. Recombinant SeV expressing luciferase was used as a control.

FIG. 6 shows the effect of RAPA treatment on the expression of angiogenic growth factors during tumor proliferation in vivo and in vitro. The relationships between tumor blood flow and angiogenic growth factors are shown for MH134 (A to C) and SAS (D).

(A and B) Reduction of blood flow in the tumor upon RAPA treatment in vivo (B: panels and a graph) and a relatively high expression pattern of murine VEGF (A). Seven days after beginning RAPA injections into mice with syngenic tumors (MH134, asterisk), the Doppler circulation image was recorded and tumor samples were subjected to ELISA. Tumors on Day 3 were also independently protein assayed (A: Day 3, n=4 in each group). On Day 7, no significant difference in the size of tumors was observed (B: asterisk).

(C) A bar graph showing that the effect of RAPA on hypoxia-induced VEGF expression in MH134 cells is significant but minimized. After 12 hours of culture under serum-free conditions, the cells were washed with fresh medium and exposed to conditions of normoxia (21% O₂) or hypoxia (2.5% O₂). After 48 hours the medium was subjected to ELISAto measure murine VEGF.

(D) RAPA-related changes in the expression of angiogenic growth factors in mice carrying a human tumor type (SAS). This observation was done to investigate the origin of the upregulated VEGF. Seven days after initiating RAPA injections to the SAS-carrying mice, tumor samples were subjected to an ELISA system specific to human and murine VEGF.

FIG. 7 shows the effect of antisense human PDGF-A gene transfer on the expression of VEGF165 from an exogenous VEGF165 gene.

FIG. 8 shows the effect of antisense human PDGF-A gene transfer on the expression of endogenous VEGF165 from tumor cells.

FIG. 9 shows the reduction in the in vivo proliferative ability of tumor cells in which PDGF-A expression has been inhibited.

FIG. 10 shows the relationship between PDGF-A mRNA and VEGF mRNA expression in fresh surgical specimens from human lung cancer.

FIG. 11 shows the relationship between the PDGF-AA-positive rate in excised human lung cancer specimens, and patient prognosis.

### BEST MODE FOR CARRYING OUT THE INVENTION

The present invention relates to methods for suppressing tumor proliferation comprising the step of inhibiting the expression of PDGF-A or the binding of PDGF-A homodimer to PDGFRα. PDGFα is a receptor for PDGF family hetero- or homodimers, including PDGF-A, -B, and -C, and activates intracellular tyrosine kinase, thereby inducing phosphorylation of itself and other downstream molecules (Claesson-Welsh, L., Prog. Growth Factor Res. 5: 37 (1994)). Activation of PDGFRα induces tumor angiogenesis via p70S6 kinase (p70S6K). p70S6 kinase is an effector molecule involved in translation of mRNA, and regulated by mTOR, a protein from the PI-kinase-related kinase (PIK-RK) family. In the present invention, the PDGFRα signal transduction pathway of mesenchymal cells was found to have an essential role not only in vascular regeneration in ischemia caused by damage and the like, but also in tumor angiogenesis. Moreover, the PDGFRα signal transduction pathway was found to be essential for tumor angiogenesis, despite the diversity of the expression patterns of angiogenic substances in each tumor type. Thus, it was concluded that in host-derived vascular systems the PDGFRα-p70S6K signal transduction pathway is a ubiquitous molecular target for inducing tumor dormancy. Furthermore, the present inventors discovered that PDGF-A in particular contributes to tumor angiogenesis, and that tumor angiogenesis can be efficiently inhibited by inhibiting PDGFRα activation by PDGF-A homodimers. Thus, inhibition of PDGF-A expression or binding between PDGF-A homodimers and PDGFRα, can inhibit formation and retention of the tumor vasculature, resulting in tumor ischemia and loss of proliferative ability and viability.

For example, reduced expression levels of PDGFRα ligands (PDGF-A, PDGF-B, PDGF-C, and such), reduced PDGFRα expression levels, reduced binding between PDGFRα and its ligands, inhibition of PDGFRα activation (a decrease in tyrosine phosphorylation level or in tyrosine kinase activity), or reduced p70S6K expression or activity can be used as indicators to confirm inhibition of the PDGFRα-p70S6K signal transduction pathway. Namely, antitumor agents can be selected by screening compounds that inhibit the above PDGFRα-p70S6K signal transduction pathway. For example, it is possible to judge whether or not expression of PDGFRα, its ligands, or p70S6 kinase has decreased by measuring the expression of these proteins or their genes (mRNAs) in the presence or absence of a test compound, and then examining whether or not expression is significantly inhibited in the presence of the test compound. In addition, to determine whether or not binding between PDGFRα and its ligands is inhibited, PDGFRα can be contacted with a ligand in the presence or absence of a test compound to examine whether or not the binding is inhibited by the test compound, for example. Tyrosine phosphorylation activity or kinase activity can be quantified by monitoring the incorporation of [γ-³²P] ATP or by using an anti-phosphorylated tyrosine antibody, or such.

Human PDGF-A gene and its encoded protein sequences are shown in Accession Nos. NM_{—}002607 (protein ID NP_{—}002598) (SEQ ID NOs: 1 and 2), NM_{—}033023 protein ID NP_{—}148983) (SEQ ID NOs: 3 and 4), protein ID AAA60045, and such (Bonthron D. T. et al., Proc. Natl. Acad. Sci. U.S.A. 85: 1492-1496 (1988); Rorsman F. et al., Mol. Cell. Biol. 8: 571-577 (1988); Betsholtz C. et al., Nature 320: 695-699 (1986); Hoppe J. et al., FEBS Lett. 223: 243-246 (1987); Takimoto Y. et al., Hiroshima J. Med. Sci. 42: 47-52 (1993); Tong B. D. et al., Nature 328: 619-621 (1987); Collins T. et al., Nature 328: 621-624 (1987); Andersson M. et al., J. Biol. Chem. 267: 11260-11266 (1992)). Other organism PDGF-As are known in, for example, rats protein ID S25096, CAA78490) (Herren, B. et al., Biochim. Biophys. Acta 1173, 294-302 (1993)), mice (Accession number NM_{—}008808, protein ID NP_{—}032834, protein ID A37359; Rorsman, F. and Betsholtz, C., Growth Factors 6, 303-313 (1992); Mercola, M. et al., Dev. Biol. 138, 114-122 (1990)), chickens (Accession number BAB62542, protein ID AB031023; Horiuchi, H. et al., Gene 272, 181-190 (2001)), and rabbits (protein ID P34007; Nakahara, K. et al., Biochem. Biophys. Res. Commun. 184, 811-818 (1992)).

Mammalian PDGF-A genes can be identified by BLAST searches or the like, based on sequences of the above-described PDGF-A genes as known PDGF-A genes (BLAST; Altschul, S. F. et al., 1990, J. Mol. Biol. 215: 403-410). Alternatively, PDGF-A genes can be obtained by RT-PCR, using primers designed based on known PDGF-A cDNAs (see Example 5). PDGF-A genes can also be readily obtained by screening cDNA libraries derived from humans, mice, rats or other mammals or birds by hybridization under stringent conditions using PDGF-A cDNAs as probes. Hybridization conditions can be determined by preparing probes from either nucleic acids comprising coding regions of PDGF-A or nucleic acids used as hybridization targets, and detecting whether the probes hybridize to other nucleic acids. Examples of stringent hybridization conditions are those where hybridization is performed in a solution containing 5×SSC (1×SSC contains 150 mM NaCl and 15 mM sodium citrate), 7% (w/v) SDS, 100 μg/ml denatured salmon sperm DNA, 5×Denhardt's solution (1×Denhardt's solution contains 0.2% polyvinyl pyrrolidone, 0.2% bovine serum albumin, and 0.2% Ficoll) at 48° C., preferably at 50° C., and more preferably at 52° C., followed by washing with shaking for two hours at the same temperature as for the hybridization, more preferably at 60° C., even more preferably at 65° C., and most preferably at 68° C. in 2×SSC, preferably in 1×SSC, more preferably in 0.5×SSC, and even more preferably in 0.1×SSC.

Nucleotide or amino acid sequences of mammalian PDGF-A generally comprise a sequence with high homology to a known PDGF-A sequence (for example, SEQ ID NOs: 1 to 4). High homology means sequence identity of 70% or more, preferably 75% or more, more preferably 80% or more, more preferably 85% or more, more preferably 90% or more, and more preferably 95% or more. Sequence identity can be determined by, for example, using the BLAST program (Altschul, S. F. et al., 1990, J. Mol. Biol. 215: 403-410). Specifically, the blastn program may be used to determine nucleotide sequence identity, while the blastx program may be used to determine amino acid sequence identity. For example, at the BLAST web page of the National Center for Biotechnology Information (NCBI), computation may be carried out using default parameters, setting the filters such as “Low complexity” to “OFF” (Altschul, S. F. et al. (1993) Nature Genet. 3:266-272; Madden, T. L. et al. (1996) Meth. Enzymol. 266:131-141; Altschul, S. F. et al. (1997) Nucleic Acids Res. 25:3389-3402; Zhang, J. & Madden, T. L. (1997) Genome Res. 7:649-656). The parameters are set, for example, as follows: open gap cost is set as 5 for nucleotides or 11 for proteins; extend gap cost is set as 2 for nucleotides or 1 for proteins; nucleotide mismatch penalty is set as -3; nucleotide match reward is set as 1; expect value is set as 10; wordsize is set as 11 for nucleotides or 2 for proteins; Dropoff (X) for blast extensions in bits is set as 20 in blastn or 7 in other programs; X dropoff value for gapped alignment (in bits) is set as 15 in programs other than blastn; and final X dropoff value for gapped alignment (in bits) is set as 50 in blastn or 25 in other programs. For amino acid sequence comparisons, BLOSUM62 can be used as a scoring matrix. The blast2sequences program (Tatiana A et al. (1999) FEMS Microbiol Lett. 174:247-250), which compares two sequences, can be used to prepare an alignment of two sequences and thus to determine their sequence identity. Identity for the entire coding sequence (CDS) of PDGF-A (for example, CDS in SEQ ID NO: 1 or 3, or SEQ ID NO: 2 or 4) is calculated by treating gaps as mismatches, and ignoring gaps outside the CDS.

In addition, polymorphisms and variants of PDGF-A can exist. For example, in human PDGF-A, variant 1, (NM_{—}002607) comprising exon 6, and variant 2 (NM_{—}033023), lacking exon 6, are known. Polymorphic forms or variants of PDGF-A can generally comprise nucleotide or amino acid sequences with substitutions, deletions, and/or insertions of one or more residues in the sequence of a certain PDGF-A molecular species (for example, CDS in SEQ ID NO: 1 or 3, or SEQ ID NO: 2 or 4). The difference from a known PDGF-A sequence is typically 30 residues or less, preferably 20 residues or less, preferably ten residues or less, more preferably five residues or less, more preferably three residues or less, and more preferably two residues or less. The amino acid substitutions may be conservative substitutions. Proteins with conservative substitutions tend to retain their activities. Conservative substitutions include, for example, amino acid substitutions among members of each group, such as basic amino acids (for example, lysine, arginine and histidine), acidic amino acids (for example, aspartic acid and glutamic acid), non-charged polar amino acids (for example, glycine, asparagine, glutamine, serine, threonine, tyrosine and cysteine), non-polar amino acids (for example, alanine, valine, leucine, isoleucine, proline, phenylalanine, methionine and tryptophan), p-branched amino acids (for example, threonine, valine and isoleucine), and aromatic amino acids (for example, tyrosine, phenylalanine, tryptophan and histidine).

Human PDGFRα gene and its encoded protein sequences are shown at Accession number NM_{—}006206 (protein ID NP_{—}006197) (SEQ ID NOs: 5 and 6), protein ID P16234, and such (Matsui T. et alt Science 243: 800-804 (1989); Claesson-Welsh L. et al., Proc. Natl. Acad. Sci. U.S.A. 86: 4917-4921 (1989); Kawagishi J. and Ku T., Genomics 30: 224-232 (1995); Strausberg R. L. et al., Proc. Natl. Acad. Sci. U.S.A. 99: 16899-16903 (2002); Cools J, et al., N. Engl. J. Med. 348: 1201-1214 (2003); Karthikeyan S. et al., J. Biol. Chem. 277: 18973-18978 (2002)). PDGFRα genes are known in other organisms, for example, mice (Accession number NM_{—}011058, protein ID NP_{—}035188) (Hamilton, T. G. et al., Mol. Cell. Biol. 23 (11), 4013-4025 (2003); Lih, C. J. et al., Proc. Natl. Acad. Sci. U.S.A. 93 (10), 4617-4622 (1996); Do, M. S. et al., Oncogene 7 (8), 1567-1575 (1992)), rats (Accession number XM_{—}214030, protein ID XP_{—}214030, P20786) (Lee, K. H. et al., Mol. Cell. Biol. 10 (5), 2237-2246 (1990); Herren, B. et al., Biochim. Biophys. Acta 1173 (3), 294-302 (1993)), and chickens (Accession number AF188842, protein ID AAF01460; Ataliotis, P., Mech. Dev. 94 (1-2), 13-24 (2000)).

Mammalian PDGFRα genes whose sequences are already known can be searched using a BLAST search or such. Alternatively, they can also be obtained by RT-PCR using primers designed based on the nucleotide sequence of a human PDGFRα or an amino acid sequence thereof (SEQ ID NOs: 5 or 6). In addition, they are also easily obtained by screening cDNA libraries from humans, mice, rats, or other mammals or avian species using known PDGFRα cDNAs as probes for hybridization under stringent conditions. The above hybridization conditions can be used. Nucleotide sequences or amino acid sequences of the PDGFRα of other organisms comprise sequences highly homologous to known PDGFRα sequences (for example, CDS of SEQ ID NO: 5 or SEQ ID NO: 6). As used herein, high homology refers to sequence identity of 70% or more, preferably 75% or more, more preferably 80% or more, more preferably 85% or more, more preferably 90% or more, and more preferably 95% or more. Identity to an entire CDS (for example, CDS of SEQ ID NO: 5 or SEQ ID NO: 6) is calculated by treating gaps as mismatches and ignoring gaps outside the CDS.

In addition, polymorphisms and variants of PDGFRα can exist. For example, polymorphisms and variants of human PDGFRα comprise substitutions, deletions, and/or insertions of one or more of residues in the CDS of SEQ ID NO: 5 or the sequence of SEQ ID NO: 6, for example. Generally residues differ by 100 residues or less, preferably 50 residues or less, more preferably 30 residues or less, more preferably ten residues or less, more preferably five residues or less, more preferably three residues or less, and more preferably two residues or less. Amino acid substitutions may be conservative substitutions.

PDGF-A expression can be inhibited by inhibiting PDGF-A transcription or translation, or by lowering the stability of PDGF-A mRNAs or PDGF-A proteins, or promoting degradation thereof. Typical methods include, for example, repressing PDGF-A expression using RNAs with RNA interference (RNAi) effect on PDGF-A genes. In general, RNAi refers to a phenomenon whereby expression of a target gene is inhibited upon destruction of a target gene mRNA, which is induced by an RNA comprising a sense RNA with a sequence homologous to a portion of the target gene mRNA sequence, and an antisense RNA with a sequence complementary thereto (Genes Dev. 2001, 15:188-200; Elbashir, S M et al., Nature 411:494-498 (2001)). When a double-stranded RNA with RNAi effect is introduced into cells, DICER, one of the RNase III nuclease family, contacts the double-stranded RNA to degrade it into small fragments called siRNAs. These siRNAs will degrade the target mRNA and repress its expression. In addition, even artificially synthesized or expressed RNA molecules, which are not RNAs generated by such intracellular processing, can function as siRNAs. In vivo methods for repressing target gene expression using siRNAs are known (Anton P. et al., Nature Vol. 418: 38-39 (2002); David L. et al., Nature Genetics Vol. 32: 107-108 (2002)).

In general, siRNAs against target genes are RNAs comprising nucleotide sequences of 15 or more contiguous bases from a transcriptional sequence (mRNA sequence) of a target gene (more preferably nucleotide sequences of 16 bases or more, 17 bases or more, 18 bases or more, or 19 bases or more), and complementary sequences thereof where these sequences form double strands upon hybridization. Preferably, siRNAs are RNAs where one strand comprises nucleotide sequences comprising 17-30 contiguous bases, more preferably sequences of 18-25 bases, more preferably sequences of 19-23 bases, or complementary sequences thereof, and where the other strand can hybridize to this strand under stringent conditions. Since in cells even RNAs comprising longer sequences are expected to be degraded to siRNAs with RNAi effect, RNA length is not thought to be limited. In addition, long chain double-stranded RNAs corresponding to full-length or virtually full-length regions of target gene mRNAs can be pre-degraded using DICER or other RNases, and these degradation products can also be used. The degradation products are expected to contain RNA molecules with RNAi effect (siRNAs). When using this method, mRNA regions expected to have RNAi effect need not be specifically selected. Namely, sequences with RNAi effect against a target gene do not necessarily require precise definition. When using synthetic siRNAs, the siRNAs can be modified appropriately.

In general, double stranded RNAs with a few bases overhang at an end are known to have strong RNAi effects. The siRNAs used in the present invention preferably have a few bases overhang at an end (preferably the 3′-end), but this is not essential. The overhang is preferably formed by two bases, but is not limited thereto. In the present invention, double-stranded RNAs comprising an overhang of, for example, TT (two thymines), UU (two uracils), or some other bases can preferably be used (most preferably molecules comprising a 19 bp double-stranded RNA portion and a two-base overhang). The siRNAs of the present invention also include such molecules where the bases forming the overhang are DNAs.

In the siRNAs, the two strands forming the base pairs may be connected via spacers. Namely, RNAs where such a spacer forms a loop, and two RNA sequences before and after the spacer anneal to form double strand, can also be suitably be used. Spacer length is not limited, but may be three to 23 bases, for example.

In addition, vectors capable of expressing the above siRNAs can also be used in the present invention. Namely, the present invention relates to uses of vectors capable of expressing RNAs with RNAi effect. The above vectors which can express RNAs may be, for example, nucleic acids where each of the strands forming a double-stranded siRNA is linked to a separate promoter, such that the two strands are separately expressed. Alternatively, two kinds of RNA may be transcribed from one promoter by alternative splicing or the like. Alternatively, the vectors may be vectors that express single-stranded RNAs where the sense and antisense strands are linked via a spacer (forming a loop). RNAs expressed from such vectors form RNA stems with RNAi effect and repress target gene expression. Stems may be, for example, 19 to 29 bases in length, which is similar to the above siRNAs. Spacers may be, for example, three to 23 bases in length, without limitation. The RNAs may or may not have a few bases overhang at the 5′ and/or 3′ end. These vectors can easily be prepared according to genetic engineering technologies standard to those skilled in the art (Brummelkamp T R et al., Science 296: 550-553 (2002); Lee N S et al., Nature Biotechnology 19: 500-505 (2001); Miyagishi M & Taira K, Nature Biotechnology 19: 497-500 (2002); Paddison P J et al., Proc. Natl. Acad. Sci. USA 99: 1443-1448 (2002); Paul C P et al., Nature Biotechnology 19: 505-508 (2002); Sui G et al., Proc Natl Acad Sci USA 99(8): 5515-5520 (2002); Proc Natl Acad Sci USA 99: 14943-14945 (2002); Paddison, P J et al., Genes Dev. 16:948-958 (2002)). More specifically, these vectors can be constructed by appropriately inserting DNAs encoding desired RNA sequences into various known expression vectors. RNA polymerase III promoters and such can be preferably used as promoters. Specifically, for example, U6 Pol III promoter and H1 RNA promoter (H1 RNA is a component of RNase P) can be used.

Examples of preferable siRNAs are shown below; however, the siRNAs used in the present invention are not limited thereto. First, a transcribed sequence region located 50 bases or more, preferably 60 bases or more, and more preferably 70 bases or more downstream of a target gene's initiation codon is selected. An AA sequence is detected in this region, and 17 to 20 nucleotides continuing from this AA (for example, 19 nucleotides continuing from MA) are selected. The base next to the AA is not especially limited, but G or C is preferably selected. Herein, the GC content of selected sequences is preferably 20% to 80%, more preferably 30% to 70%, and more preferably 35% to 65%. In addition, the selected sequences are preferably specific to a target gene among the genes expressed in tissues to which siRNAs are administered. For example, the selected sequences are preferably used as queries to search in public gene sequence databases among the genes of individuals administered with siRNAs to confirm the absence of any non-target gene that comprises the same sequence in its transcribed sequence. In addition, the sequences are preferably selected from within the protein coding sequence (CDS) regions of target genes. Sequences comprising sequences selected in this way but missing the initial MA (UU or TT is preferably added to the 3′-end) and their complementary sequences (CU or TT is preferably comprised at the 3′-end) form suitable siRNAs. It is not always necessary to search for sequences that follow on from an AA, and sequences that follow on froma CA may also be searched in the above way, for example. Alternatively, other arbitrary sequences are also acceptable. RNAs with an optimum RNAi effect can also be appropriately selected from several kinds of prepared siRNAs.

It is known that there is asymmetry in the siRNA action (Schwarz, D S. et alt, Cell 115: 199-208 (2003); Khvorova A et al., Cell, 115 (2): 209-16 (2003)). Namely, it is possible to enhance the RNAi effect against a target mRNA by selecting a sequence so that the duplex formed at the 3′-side of the sense strand (target mRNA side) of siRNA is less stable than that formed at the 5′-side. For this purpose, one to several mismatches may be introduced at the 3′-side of the sense strand.

In addition, other than siRNAs, PDGF-A expression can also be inhibited by using, for example, antisense nucleic acids against a transcriptional product of a PDGF-A gene or portions thereof, or ribozymes that specifically cleave a transcriptional product of a PDGF-A gene. Methods using antisense technology are well known to those skilled in the art as tools for inhibiting target gene expression. As detailed below, there are several factors involved in the action of antisense nucleic acids in inhibiting target gene expression. Namely, these include inhibition of transcription initiation by triplex formation, transcriptional repression by hybrid formation with a site forming a localized open loop structure by the action of RNA polymerase, transcriptional repression by hybrid formation with an RNA whose synthesis is in progress, splicing inhibition by hybrid formation at an intron-exon junction, splicing inhibition by hybrid formation with a spliceosome-forming site, inhibition of mRNA translocation from nucleus to cytoplasm by hybrid formation with the mRNA, splicing inhibition by hybrid formation with a capping site or poly (A) addition site, inhibition of translational initiation by hybrid formation with a translation initiation factor-binding site, inhibition of translation by hybrid formation with a ribosome-binding site near an initiation codon, inhibition of peptide chain elongation by hybrid formation with an mRNA translational region or a polysome-binding site, and inhibition of gene expression by hybrid formation with a nucleic acid-protein interaction site. Thus, antisense nucleic acids inhibit target gene expression by inhibiting various processes, including transcription, splicing, and translation (Hirashima and Inoue, Shin-Seikagaku Jikken Koza 2, Nucleic Acid IV Replication and Expression of Genes, The Japanese Biochemical Society Ed. Tokyo Kagaku Dojin, 1993, p. 319-347).

Antisense nucleic acids used for the present invention may inhibit PDGF-A gene expression by any of above actions. The antisense nucleic acids may be nucleic acids comprising an antisense sequence against 13 nucleotides or more, preferably 14 nucleotides or more, and more preferably 15 nucleotides or more contiguous nucleotides from a transcribed sequence of a PDGF-A gene. Preferable nucleic acids include, for example, those comprising antisense sequences against 13 nucleotides or more, preferably 14 nucleotides or more, and more preferably 15 nucleotides or more contiguous nucleotides taken from an exon-intron boundary within the early transcriptional sequence, an intron-exon boundary, a region comprising a translation initiation codon, an untranslated region near the 5′-end, or a protein-coding sequence (CDS) within a mature mRNA. In addition, when considering clinical applications, synthetic oligomers are generally used as the antisense nucleic acids. The antisense nucleic acids may be DNAs, and may also be modified. For example, S-oligos (phosphorothioate-type oligonucleotides) may be used to reduce sensitivity to nuclease digestion and to retain activity as antisense nucleic acids. In order to efficiently suppress target gene expression using antisense nucleic acids, the antisense nucleic acids are preferably 17 bases long or more, more preferably 20 bases or more, more preferably 25 bases or more, more preferably 30 bases or more, more preferably 40 bases or more, more preferably 50 bases or more, and still more preferably 100 bases or more. Antisense RNAs can also be expressed intracellularly. This is accomplished by constructing vectors that are connected to nucleic acids encoding desired RNAs downstream of promoters which are active in the target cells, and then introducing such vectors into cells.

Viral vectors such as retroviral vectors, adenoviral vectors, adeno-associated virus vectors, or minus strand RNA virus vectors, and non-viral vectors such as plasmids can be used as vectors. Use of these vector systems or gene transfer carriers (liposomes, cationic lipids, and such) enables gene therapy upon their administration to tumors.

PDGF-A gene expression can also be inhibited using ribozymes or vectors encoding ribozymes. Ribozymes refer to RNA molecules with catalytic activity. Ribozymes with a variety of catalytic activities exist, and ribozymes that cleave RNA site-specifically can also be designed. There are several types of ribozymes, including those with 400 or more nucleotides, such as group I intron types and M1 RNA comprised in RNase P, and those with around 40 nucleotide active domains (Koizumi M. and Ohtsuka E., Protein, Nucleic acid and Enzyme, 35: 2191 (1990)), such as the so called hammerhead-types (Rossi et al., Pharmac. Ther. 50: 245-254 (1991)) and hairpin-types (Hampel et al., Nucl. Acids Res. 18: 299-304 (1990), and U.S. Pat. No. 5,254,678).

For example, a self-cleaving domain of a hammerhead-type ribozyme cleaves the 3′ side of C15 in the sequence G13U14C15; however, base pair formation between U14 and A9 has been shown to be important to this activity, and sequences with A 15 or U15 instead of C15 can also be cleaved (Koizumi M. et al., FEBS Let., 228: 228 (1988)). A restriction enzyme-like RNA-cleaving ribozyme that recognizes a UC, UU or UA sequence in a target RNA can be generated by designing a ribozyme whose substrate-binding site is complementary to an RNA sequence close to a target site (Koizumi M. et al., FEBS Lett., 1988, 239: 285; Koizumi M. and Ohtsuka E., Protein, Nucleic acid and Enzyme, 35: 2191 (1990); Koizumi M. et al., Nucl Acids Res., 17: 7059 (1989)).

In addition, hairpin-type ribozymes are also useful for the objectives of the present invention. These types of ribozymes are found in, for example, the minus strands of satellite RNAs of tobacco ringspot virus (Buzayan, J M., Nature, 323: 349 (1986)). Target-specific RNA-cleaving ribozymes can be produced from hairpin-type ribozymes (Kikuchi Y. and Sasaki N., Nucl Acids Res., 19: 6751 (1991); Kikuchi Y., Kagaku to Seibutu, 30: 112 (1992)). Thus, target gene expression can be inhibited by using ribozymes to specifically cleave target gene transcripts.

When expressing ribozymes from vectors, useable vectors include viral vectors such as retroviral vectors, adenoviral vectors, adeno-associated virus vectors, and minus strand RNA virus vectors, and non-viral vectors such as plasmids.

Inhibitory effects on expression can be verified by determining mRNA levels using quantitative RT-PCR or the like, or by determining protein levels using Western blotting with an antibody or the like. Antitumor agents can be effectively screened by screening for compounds that suppress the expression of PDGF-A and/or PDGFRα. The present invention also relates to uses of compounds that suppress expression of PDGFRα or its ligands in the production of antitumor agents. In addition, the present invention relates to methods for producing antitumor agents, which comprise the step of producing compositions that comprise compounds that suppress the expression of PDGFRα or its ligands, as well as pharmaceutically acceptable carriers, and/or additives.

Moreover, binding between PDGF-AA and PDGFRα can be inhibited using, for example, compounds that bind to PDGF-AA or PDGFRα and inhibit binding between PDGF-AA and PDGFRα. The binding of PDGF-AA to a ligand can be detected by, for example, immobilizing either one to a support, contacting one with the other, and then detecting the bound substance using antibodies and such. In addition, binding can also be detected by immunoprecipitation or by pull-down assays. Alternatively, binding between PDGFRα and a ligand can also be assayed by contacting the ligand with cells expressing PDGFRα, and then detecting PDGFRα-mediated signal transduction (tyrosine phosphorylation or cell proliferation activity) and such. Antitumor agents can also be effectively screened by using these methods to measure the binding of PDGFRα to its ligands, and then screening for compounds that inhibit this binding. The present invention also relates to uses of compounds that inhibit the binding of PDGFRα to its ligands in the production of antitumor agents. In addition, the present invention also relates to methods for producing antitumor agents that comprise the step of producing compositions that comprise compounds that inhibit the binding between PDGFRα and its ligands, as well as pharmaceutically acceptable carriers, and/or additives and such.

As compounds that inhibit the binding of PDGF-AA to PDGFRα, proteins that bind to PDGFRα or its ligands and inhibit the binding between both can be produced relatively easily. More specifically, polypeptides comprising antibodies that bind to an extracellular domain of PDGFRα, or fragments of such antibodies (antibody variable regions, complementarity determining regions (CDRs), and such), polypeptides comprising antibodies that bind to PDGF-AA or fragments of such antibodies, soluble polypeptides (or secretory polypeptides) comprising a receptor-binding fragment of PDGF-A and a ligand-binding site of PDGFRα, and the like can be suitably used. The antibodies that bind to a PDGFRα extracellular domain can be produced by, for example, immunizing mammals using polypeptides comprising the PDGFRα extracellular domain or portions thereof as antigens. Alternatively, cells expressing PDGFRα or membrane fractions thereof or such may be used as antigens. As the PDGFRα extracellular domains to be used as antigens, naturally occurring soluble-type PDGFRα (Tiesman J, Hart C E., J Biol. Chem., 268 (13): 9621-8 (1993)) and artificially produced fragments comprising the extracellular domain of PDGFRα can be used. For example, a human PDGFRα amino acid sequence (SEQ ID NO: 6) from position 24 to 524, or portions thereof is preferably used as an antigen. Extracellular domains of other mammalian PDGFRα can be identified by alignment with a human PDGFRα amino acid sequence. Cell clones producing desired antibodies can be obtained by generating hybridoma cells from spleen cells, followed by selection of those hybridomas producing antibodies that bind with high affinity to an extracellular domain of PDGFRα (V. T. Oi and L. A. Herzenberg, Immunoglobulin-producing hybrid cell lines. In B. B. Misbell and S. M. Shiigi eds. Selected method in cellular immunology. pp 351-372 (1980); Iwasaki T et al., 1983, Monoclonal antibody, Hybridoma and ELISA, Kodansha Scientific, Tokyo; Toyama S, Ando T et al., ed., 1987, Monoclonal Antibody, Experimental Manual, Kodansha Scientific, Tokyo). Genes for the desired antibodies can be obtained by isolating antibody genes from the cells. By loading the genes onto vectors, vectors expressing antibodies that bind to the extracellular domain of PDGFRα can be obtained.

To obtain antibodies that bind to PDGFRα ligands, the ligands or their fragments can be used as antigens for immunizations, as above, and antibodies or their genes can be obtained. The antibodies may also be those against dimers of PDGFRα ligands. PDGFRα ligands include PDGF-A, -B, and -C, although antibodies against PDGF-A are especially preferable. For example, antibodies against PDGF-A homodimers can suitably be used.

The antibodies can be purified by, for example, ammonium sulfate precipitation, Protein A columns, Protein G columns, DEAE ion exchange columns, or antigen-coupled affinity column chromatographies. The antibodies may be polyclonal or monoclonal antibodies, so long as they bind to PDGF-A or PDGFRα, and inhibit binding between PDGF-A and PDGFRα. In addition, the antibodies may be human antibodies, antibodies humanized by genetic recombination, fragments comprising antibody variable regions (including Fab, Fc, F (ab′)2 and scFv), modified antibodies, and such. When using antibodies or antibody-expressing vectors for human administration (antibody therapies), human antibodies or humanized antibodies are preferable since they have low immunogenicity.

Antibodies that bind to PDGF-A or PDGFRα are also commercially available (for example, Rabbit anti-human PDGF-AA, Cat. No. IM-R136, DIACLONE; Anti-Human Platelet Derived Growth Factor-AA (PDGF-AA) Antibody, Leinco Technologies Inc.; Anti-PDGF-AA neutralizing goat antibody, R&D systems; Anti-PDGFRα neutralizing goat antibody, R&D systems).

Secretory proteins comprising an extracellular domain of PDGFRα can be suitably used as secretory polypeptides comprising a PDGFRα ligand-binding site. Such proteins are also known to exist in nature (Tiesman J, Hart C E., J Biol. Chem., 268 (13): 9621-8 (1993)). Alternatively, artificially produced secretory proteins comprising an extracellular domain of PDGFRα can be used (see Examples). The PDGFRα extracellular domain has five immunoglobulin (Ig)-like domains, the first three domains of which (domains 1 to 3) (the human PDGFRα amino acid sequence (SEQ ID NO: 6) from position 24 to 341) are known to have ligand-binding activity (D. Mahadevan et al., J. Biol. Chem., 270, 27595-27600 (1995); B Herren et al., J. Biol. Chem., 268, 15088-15095 (1993)). Thus, by using secretory proteins comprising these three Ig-like regions, and preferably comprising the five Ig-like regions (the human PDGFRα amino acid sequence (SEQ ID NO: 6) from position 24 to 524), PDGF-AA is absorbed and its binding to the endogenous receptor can be inhibited. Appropriate secretory signal sequences can be added to the N-terminus of the proteins to enable their secretion. For example, the amino acid sequence from position 1 to 23 of human PDGFRα can be used as a secretory signal sequence, and soluble proteins comprising an amino acid sequence from position 1 to 524 of human PDGFRα can be suitably used. The PDGFRα extracellular domains of other mammals can be identified by alignment with an amino acid sequence of human PDGFRα.

To express the above proteins by vector-mediated gene therapy, vectors carrying nucleic acids encoding the above proteins can be constructed by recombinant gene technology. Herein, “encoding a protein” means that a nucleic acid comprises an ORF encoding an amino acid sequence of a protein in a sense or antisense (in certain types of viral vectors) orientation, such that the protein can be expressed under appropriate conditions. The nucleic acids may be single- or double-stranded, depending on the type of vector. Further, the nucleic acids can be DNAs or RNAs. The vectors include, for example, plasmid vectors, other naked DNAs, and viral vectors.

Naked DNAs refer to DNAs not bound to reagents for introducing nucleic acids into cells, such as viral envelope, liposomes, or cationic lipids (Wolff et al., Science 247: 1465-1468 (1990)). In such cases, the DNAs can be used upon dissolution in a physiologically acceptable solution, for example, sterile water, physiological saline, or a buffer. Injection of naked DNAs such as plasmids is the safest and most convenient gene delivery method, and is used in the many clinical protocols approved so far (Lee, Y. et al., Biochem. Biophys. Res. Commun. 272: 230-235 (2000)). For example, the cytomegalovirus (CMV) promoter is one of the strongest transcriptional regulatory sequences available, and vectors comprising the CMV promoter are also widely used in clinical gene therapy (Foecking, M. K, and Hofstetter H. Gene 45: 101-105 (1986)). In addition, a suitably used promoter is CAG promoter (Niwa H. et al., Gene. 108: 193-199 (1991)), which is a chimeric promoter comprising CMV immediately early enhancer and chicken β-actin promoter, and which enables expression stronger than or equal to CMV promoter.

When integrating desired genes into vectors, a Kozak consensus sequence (for example, CC (G/A) CCATG) is preferably used near the initiation codon to enhance the expression efficiency of the desired genes (Kozak M., Nucleic Acids Res 9 (20): 5233 (1981); Kozak M., Cell 44: 283 (1986); Kozak M., Nucleic Acids Res. 15: 8125 (1987); Kozak M., J. Mol. Biol. 196; 947 (1987); Kozak M., J. Cell Biol. 108: 229 (1989); Kozak M., Nucl. Acids Res. 18: 2828 (1990)).

DNAs can be appropriately administered in combination with transfection reagents. For example, transfection efficiency can be enhanced by binding DNAs to liposomes or to desired cationic lipids.

Viral vectors are more preferable vectors for use in the present invention. Use of viral vector allows expression of sufficient amounts of polypeptides in a wide range of tissues. Viral vectors include adenoviral vectors, adeno-associated virus vectors, retroviral vectors, lentivirus vectors, herpes simplex virus vectors, vaccinia virus vectors, and minus strand RNA virus vectors, but are not limited thereto. A preferable vector is an adenoviral vector. Adenoviral vectors can very efficiently introduce genes into a wide range of tissues, allowing strong expression of introduced genes, Adenoviral vectors are preferably used in the present invention. In the present invention, known adenoviral vectors can be appropriately used. Wild-type adenovirus genes contained in the vectors may be altered, for example, to increase exogenous gene expression or reduce immunogenicity. When constructing adenoviral vectors, the COS-TPC method developed by Saito et al., for example, can be used (Miyake S., Proc. Natl. Acad. Sci. USA 93: 1320-1324 (1996)).

Other viral vectors suitably used in the present invention are minus strand RNA virus vectors. As shown in Examples, gene therapy using minus strand RNA virus vectors significantly suppressed in vivo tumor proliferation. Minus strand RNA virus vectors are some of the most suitable vectors for use in the present invention. Herein, a “minus-strand RNA virus” refers to a virus that includes a minus strand RNA (an antisense strand corresponding to a sense strand encoding a viral protein) as the genome. The minus-strand RNA is also referred to as a negative strand RNA. The minus-strand RNA viruses used in the present invention particularly include single-stranded minus-strand RNA viruses (also referred to as non-segmented minus-strand RNA viruses). A “single-strand negative strand RNA virus” refers to viruses having a single-stranded negative strand RNA (i.e., a minus strand) as the genome. Such viruses include viruses belonging to Paramyxoviridae (including the genera Paramyxovirus, Morbillivirus, Rubulavirus, and Pneumovirus), Rhabdoviridae (including the genera Vesiculovirus, Lyssavirus, and Ephemerovirus), Filoviridae, Orthomyxoviridae, (including Influenza viruses A, B, and C, and Thogoto-like viruses), Bunyaviridae (including the genera Bunyavirus, Hantavirus, Nairovirus, and Phlebovirus), Arenaviridae, and the like. The minus strand RNA virus vectors used in the present invention may be transmissible, or may be deficient-type vectors without transmission ability. The term “transmissible” means that when host cells are infected with the viral vector, the virus replicates within the cells, and infective virus particles are produced.

In particular, minus strand RNA virus vectors encoding secretory proteins that comprise antibodies that bind to PDGF-A, antibodies that bind to an extracellular domain of PDGFRα, or antigen-binding fragments thereof; and minus strand RNA virus vectors encoding secretory proteins comprising a ligand-binding domain of PDGFRα, are useful as antitumor agents of the present invention. By directly or indirectly administering these vectors to tumors, it is possible to suppress tumor proliferation. For indirect administration, the vectors can be introduced into dendritic cells, and the cells are then administered to tumors, for example.

Minus-strand RNA viruses preferably used in the present invention include, for example, Sendai viruses, Newcastle disease viruses, mumps viruses, measles viruses, respiratory syncytial viruses (RS virus), rinderpest viruses, distemper viruses, simian parainfluenza viruses (SV5), and human parainfluenza viruses 1, 2, and 3 belonging to Paramyxoviridae; influenza viruses belonging to Orthomyxoviridae; and vesicular stomatitis viruses and rabies viruses belonging to Rhabdoviridae.

Further examples of the viruses that may be used in the present invention include: Sendai viruses (SeV), human parainfluenza virus-1 (HPIV-1), human parainfluenza virus-3 (HPIV-3), phocine distemper viruses (PDV), canine distemper viruses (CDV), dolphin molbillivirus (DMVM), peste-des-petits-ruminants virus (PDPR), measles viruses (MV), rinderpest viruses (RPV), Hendra viruses (Hendra), Nipah viruses (Nipah), human parainfluenza virus-2 (HPIV-2), simian parabifluenza virus 5 (SV5), human parainfluenza virus-4a (HPIV-4a), human parainfluenza virus-4b (HPIV-4b), mumps viruses (Mumps), and Newcastle disease viruses (NDV). More preferable examples are virus selected from the group consisting of Sendai viruses (SeV), human parainfuenza virus-1 (HPIV-1), human parainfluenza virus-3 (HPIV-3), phocine distemper viruses (PDV), canine distemper viruses (CDV), dolphin molbillivirus (DMV), peste-des-petits-ruminants virus (PDPR), measles viruses (MV), rinderpest viruses (RPV), Hendra viruses (Hendra), and Nipah viruses (Nipah).

More preferably, the viruses used in the present invention are those belonging to Paramyxoviridae (including Respirovirus, Rubulavirus, and Morbillivirus) or derivatives thereof, and more preferably, those belonging to the genus Respirovirus (also referred to as Paramyxovirus) or derivatives thereof. The derivatives include viruses that are genetically-modified or chemically-modified so as not to impair their ability to transfer genes. Examples of viruses of the genus Respirovirus applicable to the present invention are human parainfluenza virus-1 (HPIV-1), human parainfluenza virus-3 (BPIV-3), bovine parainfluenza virus-3 (BPIV-3), Sendai virus (also referred to as murine parainfluenza virus-1), and simian parainfluenza virus-10 (SPIV-10). A more preferred paramyxovirus in the present invention is a Sendai virus. These viruses may be derived from natural strains, wild strains, mutant strains, laboratory-passaged strains, artificially constructed strains, or the like.

Recombinant minus strand RNA virus vectors can be reconstituted using known methods (WO97/16539; WO97/16538; WO00/70055; WO00/70070; WO03/025570; PCT/JP03/07005; PCT/JP2004/000944; Durbin, A. P. et al., 1997, Virology 235: 323-332; Whelan, S. P. et al., Proc. Natl. Acad. Sci. USA 92: 8388-8392 (1995); Schnell. M. J. et al., EMBO J. 13: 4195-4203; (1994) Radecke, F. et alt, EMBO J. 14: 5773-5784 (1995); Lawson, N. D. et al., Proc. Natl. Acad. Sci. USA 92: 4477-4481; Garcin, D. et al., EMBO J. 14: 6087-6094 (1995); Kato, A. et al., Genes Cells 1: 569-579 (1996); Baron, M. D. and Barrett, T., J. Virol. 71: 1265-1271 (1997); Bridgen, A. and Elliott, R. M., Proc. Natl. Acad. Sci. USA 93: 15400-15404 (1996); Hasan, M. K. et al., J. Gen. Virol. 78: 2813-2820 (1997); Kato, A. et al., EMBO J. 16: 578-587 (1997); Yu, D. et al., Genes Cells 2: 457-466 (1997)). These methods enable the reconstitution of minus strand RNA viruses including parainfluenza virus, vesicular stomatitis virus, rabies virus, measles virus, rinderpest virus, and Sendai virus from DNA. The minus strand RNA viruses of the present invention can be reconstituted by following these methods. For DNAs encoding a viral genome, deletion from the virus genome of the F, HN, and/or M genes and such, which encode proteins that constitute the envelope, will prevent formation of infectious virus particles; however, it is possible to generate infectious virus particles by separately introducing these deleted genes and/or genes encoding envelope proteins from another virus (for example, the vesicular stomatitis virus (VSV) G protein (VSV-G) (J. Virology 39: 519-528, 1981)) into host cells and then expressing them (Hirata, T. et al., J. Virol. Methods, 104: 125-133 (2002); Inoue, M. et al., J. Virol. 77:6419-6429 (2003)).

Minus-strand RNA viruses do not have a DNA phase and only carry out transcription and replication in the host cytoplasm; consequently, chromosomal integration does not occur (Lamb, R. A. and Kolaokofsky, D., Paramyxoviridae: The viruses and their replication. In: Fields B N, Knipe D M, Howley P M, (eds). Fields Virology, 3rd Edition, Vol. 2. Lippincott-Raven Publishers: Philadelphia, 1996, pp. 1177-1204). Thus, problems with safety such as transformation and immortalization due to chromosomal abberation do not occur. This characteristic of minus-strand RNA viruses greatly contributes to safety when they are used as vectors. For example, in the results of foreign gene expression almost no nucleotide mutation is observed, even after multiple continuous passaging of SeV, suggesting that the viral genome is highly stable and the inserted foreign genes are stably expressed over long periods of time (Yu, D. et al., Genes Cells 2, 457-466 (1997)). Further, since SeV does not have a capsid structural protein, there are qualitative advantages such as flexibility in packaging or inserted gene size. Further, SeV are known to be pathogenic in rodents, causing pneumonia, but are not confirmed as human pathogens. This is supported by previous reports that nasal administration of wild type SeV to non-human primates does not show severe harmful effects (Hurwitz, J. L. et al., Vaccine 15: 533-540, 1997; Bitzer, M. et al., J. Gene Med, 5: 543-553, 2003). Minus-strand RNA viral vectors are extremely useful as vectors that can be used in the present invention.

The recovered viral vectors can be purified to be substantially pure. Purification can be achieved using known purification/separation methods, including filtration, centrifugation, adsorption, and column purification, or any combinations thereof. “Substantially pure” means that a major proportion of a solution containing a viral vector is the viral component. For example, a viral vector composition can be confirmed to be substantially pure if the proportion of protein contained as the viral vector component as compared to total protein (excluding proteins added as carriers and stabilizers) in the solution is 10% (w/w) or greater, preferably 20% or greater, more preferably 50% or greater, preferably 70% or greater, more preferably 80% or greater, and even more preferably 90% or greater. Specific purification methods for paramyxovirus vectors for example, include methods using cellulose sulfate esters or cross-linked polysaccharide sulfate esters (Japanese Patent Application Kokoku Publication No. (JP-B) S62-30752 (examined, approved Japanese patent application published for opposition), JP-B S62-33879, and JP-B S62-30753) and methods including adsorption to fucose sulfate-containing polysaccharides and/or degradation products thereof (WO97/32010), but are not limited thereto.

Tumor proliferation is suppressed by administering to tumors compounds, nucleic acids, or proteins that inhibit the expression of PDGF-A or the binding between PDGF-A homodimers and PDGFRα, as mentioned above, or vectors expressing them. Herein, “administering to tumors” means administering to the interior or vicinity of a tumor in such a way as to inhibit formation and/or retention of the tumor vasculature. “Vicinity” is a region sufficiently close to the tumor, where blood supply to the tumor can be significantly reduced upon destruction of the vasculature in the administered region. In general, the region is within 9 mm, preferably within 8 mm, more preferably within 7 mm, more preferably within 6 mm, more preferably within 5 mm, and more preferably within 3 mm from the tumor. The administered substances or expression products from the administered vectors inhibit PDGFRα-p70S6 kinase signal transduction, thereby inhibiting formation and retention of the vasculature in the vicinity of the tumors. This leads to interception of blood supply to the tumor, resulting in suppression of tumor proliferation. The administered compounds or vectors can be administered as compositions in combination with carriers. The carriers to be used are not limited so long as they are physiologically acceptable, and include organic substances such as biopolymers, inorganic substances such as hydroxyapatites, and specifically include collagen matrices, polylactic acid polymers or copolymers, polyethylene glycol polymers or copolymers, and their chemical derivatives. Moreover, the carriers may also be mixed compositions with these physiologically acceptable materials. When administering vectors, desired vectors can be used, including viral and non-viral vectors. When expressing secretory proteins from vectors, vectors may be administered in the form of cells to which the vectors have been introduced (ex vivo administration). For example, tumors may be injected with vectors, or cells to which vectors have been introduced. For example, dendritic cells (DCs) are preferable as the cells. Examples of the injection tools include industrial products such as conventional medical syringes or ex vivo/in vivo continuous infusion devices.

When dendritic cells are used for ex vivo administration, for example, lymphocytic dendritic cells (including cells which induce Th2 or immune tolerance), bone marrow dendritic cells (generally used dendritic cells, including immature and mature dendritic cells), Langerhans cells (dendritic cells important as antigen-presenting cells in the skin), interdigitating cells (distributed in the lymph nodes and spleen T cell region, and believed to function in antigen presentation to T cells), and follicular dendritic cells (important as antigen-presenting cells for B cells; these cells present antigens to B cells by presenting antigen-antibody complexes or antigen-complement complexes on the surface via the antibody receptor or the complement receptor) can be used. The dendritic cells are, for example, CD1a⁺, HLA-class II⁺, and CD11c⁺ cells that do not express T cell marker (CD3), B cell markers (CD19, CD20), NK cell marker (CD56), neutrophil marker (CD15), and monocyte marker (CD14). Seethe following references regarding expression of these marker genes (Knapp, W. et al., eds., 1989, Leucocyte Typing IV: White Cell Differentiation Antigens, Oxford University Press, New York; Barclay, N. A. et al., eds., 1993, The Leucocyte Antigen Facts Book, CD11 Section, Academic Press Inc., San Diego, Calif., p. 124; Stacker, S. A. and T. A. Springer, 1991, J. Immunol. 146:648; Knapp, W. et al., eds., 1989, Leucocyte Typing IV: White Cell Differentiation Antigens, Oxford University Press, New York; Schlossman, S. et al., eds., 1995, Leucocyte Typing V: White Cell Differentiation Antigens. Oxford University Press, New York; Hanau, D. et al., 1990, J. Investigative Dermatol. 95: 503; Calabi, F. and A. Bradbury., 1991., Tissue Antigens 37: 1; McMichael, A. J. et al., eds., 1987, Leucocyte Typing III: White Cell Differentiation Antigens, Oxford University Press, New York; Knapp, W. et al., eds., 1989, Leucocyte Typing IV: White Cell Differentiation Antigens, Oxford University Press, New York; Schlossman, S. et al., eds., 1995, Leucocyte Typing V: White Cell Differentiation Antigens. Oxford University Press, New York; Wright, S. D. et al., 1990, Science 249:1434; Pawelec, G. et al., 1985, Human Immunology 12:165; Ziegler, A. et al., 1986, Immunobiol. 171:77). Antibodies to such markers are commercially available, for example, from BD Biosciences (BD PharMingen), and detailed information is available at the websites of the company or its distributors.

For dendritic cell markers, see also references by Kiertscher et al. and Oehler et al. (Kiertscher S M, Roth M D, Human CD14⁺ leukocytes acquire the phenotype and function of antigen-presenting dendritic cells when cultured in GM-CSF and IL-4, J. Leukoc. Biol., 1996, 59(2):208-18; Oehler, L. et al., Neutrophil granulocyte-committed cells can be driven to acquire dendritic cell characteristics., J. Exp. Med., 1998, 187(7):1019-28). The expression of each of the markers may be determined, for example, by staining with an isotype control antibody and using the fluorescence intensity for a positive rate of 1% or less as a threshold, wherein fluorescence equal to or above the threshold is deemed positive, and fluorescence below is deemed negative.

Dendritic cells or precursor cells thereof can be prepared according to or based on known methods. For example, the cells can be isolated from blood (for example, peripheral or cord blood), bone marrow, lymph nodes, other lymphatic organs, spleen, skin, and so on. The dendritic cells to be used in the present invention are preferably obtained from blood or bone marrow. Alternatively, the dendritic cells to be used in the present invention may be skin Langerhans cells, veiled cells of afferent lymphatics, follicular dendritic cells, spleen dendritic cells, and interdigitating cells of lymphatic organs. The dendritic cells used in the present invention include dendritic cells selected from the group consisting of CD34⁺-derived dendritic cells, bone marrow-derived dendritic cells, monocyte-derived dendritic cells, splenic cell-derived dendritic cells, skin-derived dendritic cells, follicular dendritic cells, and germinal center dendritic cells. CD34⁺-derived dendritic cells can be differentiated from hematopoietic stem cells, hematopoietic progenitor cells, or the like, obtained from cord blood, bone marrow, or the like, using granulocyte colony stimulating factor (G-CSF), granulocyte macrophage colony stimulating factor (GM-CSF), tumor necrosis factor (TNF)-alpha, IL-4, IL-13, stem cell factor (SCF), Flt-3 ligand, c-kit ligand, combinations thereof, or the like. For example, peripheral blood monocytes can be differentiated into immature dendritic cells using GM-CSF and IL-4, and further differentiated into mature dendritic cells by stimulation with TNF-alpha.

Specific methods for isolating dendritic cells are described in, for example, Cameron et al., Science 257:383 (1992); Langhoff et al., Proc. Natl. Acad. Sci. USA 88:7998 (1991); Chehimi et al., J. Gen. Virol. 74:1277 (1993); Cameron et al., Clin. Exp. Immunol. 88:226 (1992); Thomas et al, 1993, J. Immunol. 150:821 (1993); and Karhumaki et al., Clin. Exp. Immunol. 91:482 (1993). The isolation of dendritic cells by flow cytometry is described in, for example, Thomas et al, J. Immunol. 153:4016 (1994); Ferbas et al., J. Immunol. 152:4649 (1994); and O'Doherty et al. Immunology 82:487 (1994). In addition, magnetic cell separation is described in, for example, Miltenyi et al., Cytometry 11: 231-238 (1990).

Furthermore, for example, human dendritic cells may be isolated and grown using the methods described in Macatonia et al., Immunol. 74:399-406 (1991); O'Doherty et al., J. Exp. Med. 178:1067-1078 (1993); Markowicz et al., J. Clin. Invest. 85:955-961 (1990); Romani et al., J. Exp. Med. 180:83-93 (1994); Sallusto et al., J. Exp. Med. 179:1109-1118 (1994); Berhard et al., J. Exp. Med. 55:1099-1104 (1995); and the like. Moreover, dendritic cells can be formed from CD34⁺ cells obtained from bone marrow, cord blood, peripheral blood, or the like and from peripheral blood-derived mononuclear cells by the method described in Van Tendeloo et al., Gene Ther. 5:700-707 (1998).

Doses of the antitumor agents described herein may vary depending on patient body weight, age, sex and symptoms, the form of the composition to be administered, the administration methods, and so on, and doses can be appropriately determined by those skilled in the art. The frequency of administration is one or more times, within the range of clinically acceptable side effects. Administration may also be to one or more sites. When administered orally, adult doses of non-peptide low molecular weight compounds are generally within the range of about 0.1 to 100 mg per day, preferably about 1.0 to 50 mg per day, and more preferably about 1.0 to 20 mg per day (for 60 kg in body weight). When administered parenterally, doses vary depending on the subject to be administered, the target organ, symptoms, and administration route, but doses can be injected intravenously when administered in injectable forms, and range from, for example, about 0.01 to 30 mg per day, preferably about 0.1 to 20 mg per day, and more preferably about 0.1 to 10 mg per day. For other animals, for example, the doses can be calculated by correcting the above doses for weight. The doses of protein formulations will range from about 100 μg to 50 mg per day, for example. For example, the administration site for viral vectors may be one or more sites (for example, two to ten sites) inside or in the vicinity of the tumor. Preferable doses of adenoviruses are, for example, 10¹⁰ to 10¹³ pfu, more preferably 10¹¹ to 10¹³ pfu. The preferable doses of minus strand RNA viruses are, for example, 2×10⁵ CIU to 5×10¹¹ CIU. The administration sites for naked DNAs, antisense nucleic acids, siRNAs, or such, may be one or more sites (for example, two to ten sites) inside or in the vicinity of the tumor. Preferable doses per site are, for example, 10 μg to 10 mg, and more preferably 100 μg to 1 mg. When vector-introduced cells are administered ex vivo, for example, the viral vectors are introduced into target cells outside the body (for example, in test tubes or in dishes) at an MOI of one to 500. The transgenic cells can be transplanted into tumors at doses of 10⁵ to 10⁹ cells, and preferably 10⁶ to 10⁸ cells. The document Freedman S B et al Ann Intern Med, 136; 54-71 (2002) can be referred to regarding doses. Animal subjects for the treatments include humans and other desired non-human animals, specifically humans, monkeys, mice, rats, rabbits, sheep, cattle, and dogs.

The present invention also relates to antitumor agents comprising compounds that inhibit the expression of PDGF-A or the binding of PDGF-A homodimers to PDGFRα as active ingredients. In addition, the present invention relates to uses of the compounds that inhibit the expression of PDGF-A or the binding of PDGF-A homodimers to PDGFRα in the production of antitumor drugs. Herein, examples of the above compounds include antisense RNAs and siRNAs of PDGF-A genes, and vectors encoding the antisense RNAs or siRNAs. Further, the compounds include secretory proteins that bind to PDGF-A homodimers or PDGFRα, or vectors encoding the secretory proteins. Such secretory proteins include antibodies that bind to PDGF-A homodimers or PDGFRα, their fragments, and soluble PDGFRα. As the vectors, for example, minus strand RNA virus vectors can suitably be used. The vectors are preferably formulated into injectable forms and such for local administration to tumors.

The above-mentioned antitumor agents may be compositions comprising pharmaceutically acceptable carriers and/or additives, in addition to the active ingredients. For example, they may comprise sterile water, physiological saline, conventional buffers (phosphate, citrate, other organic acids, and such), stabilizers, salts, antioxidants (ascorbic acid and the like), surfactants, emulsifiers, isotonic agents, or preservatives. Combination with organic substances such as biopolymers, inorganic substances such as hydroxyapatites, and specifically collagen matrices, polylactic acid polymers or copolymers, polyethylene glycol polymers or copolymers, or their chemical derivatives, is also preferable for local administration. When preparing formulations suitable for injection, the active ingredients are dissolved in pharmaceutically acceptable aqueous solutions or prepared as lyophilized formulations that can be dissolved, for example. In addition, the active ingredients may be combined as kits with carriers used for dissolution or dilution. Such carriers include pharmaceutically acceptable carriers, for example, distilled water and physiological saline.

### EXAMPLES

Herein below, the present invention will be specifically described with reference to Examples, but it is not to be construed as being limited thereto. In addition, the references cited herein are incorporated as a part of this specification.

### Cells and Reagents

HSMC (J. Cell Biol., 50: 172-86 (1971)), MRC-5 (ATCC CCL-171), SAS (J. Biol. Chem., 270 (41): 24321-69 (1995)), MH134 (J. Natl. Cancer Inst., 17: 1-21 (1956)), QG56 (Int. J. Cancer, 35 (6): 808-12 (1985)), TF (Cancer, 69 (10): 2589-97 (1992)), KN (Cancer, 69 (10): 2589-97 (1992)), EBC-1 (Am. J. Pathol., 142 (2): 425-31 (1993)), PC9 (Int. J. Cancer, 15 (4): 449-55 (1985)), and COS7 Cells (ATCC CRL-1651) were purchased from American Type Culture Collection (ATCC). As mentioned previously, the intracellular signal inhibitors below were each used at the following concentrations for HSMC and MRC5 cells (Onimaru M et al., Circ Res. 91: 723-730 (2002)): Ras, Ras-inhibitory peptide (50 μmol/L, Alexis Japan, Tokyo, Japan); p70S6K, p70S6K inhibitor rapamycin (100 ng/ml, Sigma-Aldrich Japan, Tokyo, Japan); PKC, PKC inhibitor bisindolylmaleimide (100 nmol/L, Sigma); PI3K, PI3K-inhibitor wortmannin (120 nmol/L, Sigma); MEK inhibitor U0126 (10 μmol/L, Promega K.K., Tokyo, Japan); PKA, PKA-inhibitory peptide (1 μmol/L, Calbiochem, San Diego, Calif.); and NF-κG, NF-κB inhibitor ALLN (5 μmol/L, Roche Diagnostics, Tokyo, Japan).

Anti-PDGF-AA-neutralizing goat antibody, anti-PDGFRα neutralizing goat antibody, and control goat IgG were purchased from R&D systems (Minneapolis, Minn.). The stocks of recombinant SeVs, including mouse FGF-2-encoding SeV (SeV-FGF2) and firefly luciferase-encoding SeV (SeV-luciferase) used in the present invention were prepared as mentioned previously (Masaki I et al., Circ Res. 90: 966-973 (2002); Onimaru M et al., Circ Res. 91: 723-730 (2002)). Recombinant SeV expressing the extracellular domain of human PDFGRα was constructed as follows: Total RNA was extracted from MRC-5 cells; cDNA was then synthesized from this total RNA by reverse transcription and used as a template to amplify cDNA fragments (amplified region: position 1-1575 bases of CDS) using synthetic primers with restriction enzyme site tags (foward-BglII: 5′-aaAGATCTatggggacttcccatcggc-3′ (SEQ ID NO: 9) and reverse-NheI: 5′-ttGCTAGCtcacttgtcatcgtcgtccttgtagtcttcagaacgcagggt-3′ (SEQ ID NO: 10); and the obtained cDNA fragments were subcloned into pcDNA3.1 (Invitrogen, Carlsbad, Calif.) (SEQ ID NOs: 7 and 8). Clones whose entire sequence was confirmed by capillary sequencer (model CEQ2000L, Beckman Coulter Inc., Fullerton, Calif.) to be completely identical to a reported known sequence (GenBank No. NM_{—}006206) were subcloned into the template plasmid encoding SeV18+ (Hasan, M. K. et al., J. Gen. Virol. 78: 2813-2820 (1997)). Recombinant SeV (SeV-hsPDGFRα) expressing soluble human PDGFRα was recovered, as mentioned previously (Masaki I et al., Circ Res. 90: 966-973 (2002); Onimaru M et al., Circ Res. 91: 723-730 (2002); Yonemitsu Y et al., Nat. Biotechnol. 18: 970-973 (2000)). Soluble human PDGFRα was confirmed by Western blotting to be secreted into the culture supernatant of COS7 cells to which SeV-hsPDGFRα had been introduced (data not shown).

### Animals

Male C57BL/6 mice (six weeks old) and balb/c nu/nu mice (five weeks old) were purchased from KBT Oriental Co., Ltd. (Charles River Grade, Tosu, Saga, Japan). All animal experiments were carried out using approved procedures and in accordance with recommendations for the proper care and use of laboratory animals by the Committees for Animal, Recombinant DNA, and Infectious Pathogen Experiments at Kyushu University and according to The Law (No. 105) and Notification (No. 6) of the Japanese Government.

### Limb Ischemia Model

Details of surgical procedures and limb prognosis evaluation are described (Masaki I et al, Circ Res. 90: 966-973 (2002); Onimaru M et al., Circ Res. 91: 723-730 (2002)). For gene transfer, 25 μl of vector solution was injected into two portions of femoral muscle, immediately after the operation. Endogenous PDGF-AA activity was suppressed in vivo using PDGF-AA-specific neutralizing goat polyclonal IgG (cross-reactive to human and mouse proteins) (R&D) via a disposable micro-osmotic pump (Model 1007D, ALZA Co., Mountain View, Calif.), as described previously (Masaki I et al., Circ Res. 90: 966-973 (2002); Onimaru M et al., Circ Res. 91: 723-730 (2002)).

### Tumor Implantation

10⁶ SAS or MH134 cells were implanted into the abdominal wall endothelium, and tumor volume was assessed every other day. Seven days after implantation, RAPA (1.5 mg/kg/day) was administered intraperitoneally every day. Mice were sacrificed on Day 7 or Day 28, and the tumors were subjected to ELISA.

### Enzyme-Linked Immunosorbent Assay (ELISA)

As mentioned previously (Masaki I et al., Circ Res. 90: 966-973 (2002); Onimaru M et al., Circ Res. 91: 723-730 (2002)), the protein contents of the mouse limb muscle, tumor, and culture medium were determined using Quantikine Immunoassay systems for mice (both the 164 and 220 amino acid residue forms are recognized) and human VEGF-A, human FGF-2 (available to both human and mouse), human HGF (R&D Systems Inc., Minneapolis, Minn.), and rat HGF (available to mouse HGF; Institute of Immunology Inc., Tokyo, Japan), according to the manufacturer's instructions.

### Northern Blot Analysis

Total cellular RNA, isolated using the ISOGEN system (Wako Pure Chemicals, Osaka, Japan), was electrophoresed and transferred onto a nylon membrane. The membranes were hybridized overnight at 60° C. with random-primed [α-³²P]dCTP-labeled probes. The bands were visualized and subjected to densitometry using a photoimager.

### Real-Time PCR

Total RNA was extracted from the ischemic limb muscles using the ISOGEN system, then treated with RNase-free DNase (Boehringer), Dispensed total RNA (25 ng) was reverse transcribed, and then amplified in triplicate using a TaqMan EZ RT-PCR kit and Sequence Detection System, model 7000 (PE Biosystems), according to the manufacturers' protocols. The table shows the nucleotide sequences of the PCR primers and TaqMan probes. The mouse GAPDH control reagent was used as an internal standard. Target amounts were determined from a relative standard curve constructed using serial dilutions of the control total RNA (PE Biosystems), according to manufacturer's instructions. The expression levels of the target gene in each of the samples were normalized using the expression levels of GAPDH.

TABLE 1
Nucleotide sequences of primers and probes used for real-time PCR

VEGF (amplicon size: 137 bp)

| | | |
|---|---|---|
| VEGF-forward | 5′-GCAGGCTGCTGTAACGATGAA-3′ | (SEQ ID NO: 11) |
| VEGF-reverse | 5′-TCACATCTGCTGTGCTGTAGGA-3′ | (SEQ ID NO: 12) |
| VEGF-hybridi- | 5′-FAM-CATGCAGATCATGCGGATCAAACCTC-TAMRA-3′ | (SEQ ID NO: 13) |
| zation probe | | |

HGF (amplicon size: 87 bp)

| | | |
|---|---|---|
| HGF-forward | 5′-CAGCAATACCATTTGGAATGGAAT-3′ | (SEQ ID NO: 14) |
| HGF-reverse | 5′-TTGAAGTTCTCGGGAGTGATATCA-3′ | (SEQ ID NO: 15) |
| HGF-hybridi- | 5′-FAM-CGTTGGGATTCGCAGTACCCTCACA-TAMRA-3′ | (SEQ ID NO: 16) |
| zation probe | | |

PDGF-A (amplicon size: 125 bp)

| | | |
|---|---|---|
| PDGF-A-forward | 5′-CGTCAAGTGCCAGCCTTCA-3′ | (SEQ ID NO: 17) |
| PDGF-A-reverse | 5′-ATGCACACTCCAGGTGTTCCT-3′ | (SEQ ID NO: 18) |
| PDGF-A-hybridi- | 5′-FAM-CACTTTGGCCACCTTGACACTGCG-TAMRA-3′ | (SEQ ID NO: 19) |
| zation probe | | |

PDGFRα (amplicon size: 148 bp)

| | | |
|---|---|---|
| PDGFR α-forward | 5′-GAGCATCTTCGACAACCTCTACAC-3′ | (SEQ ID NO: 20) |
| PDGFRα-reverse | 5′-CCGGTATCCACTCTTGATCTTATTG-3′ | (SEQ ID NO: 21) |
| PDGFRα-hybridi- | 5′-FAM-CCCTATCCTGGCATGATGGTCGATTCT-TAMRA-3′ | (SEQ ID NO: 22) |
| zation probe | | |

GAPDH (amplicon size: 117 bp)

| | | |
|---|---|---|
| GAPDH-forward | 5′-CCTGGAGAAACCTGCCAAGTAT-3′ | (SEQ ID NO: 23) |
| GAPDH-reverse | 5′-TTGAAGTCGCAGGAGACAACCT-3′ | (SEQ ID NO: 24) |
| GAPDH-hybridi- | 5′-FAM-TGCCTGCTTCACCACCTTCTTGATGT-TAMRA-3′ | (SEQ ID NO: 25) |
| zation probe | | |

### Laser-Doppler Perfusion Images

As mentioned previously, blood flow in the tumors was assessed using a Laser Doppler perfusion image (LDPI) analyzer (Moor Instruments, Devon, UK) (Masaki I et al., Circ Res. 90: 966-973 (2002); Onimaru M et al., Circ Res. 91: 723-730 (2002)). To remove background noise from blood flow in the small intestine, a blue-sheet was inserted into the peritoneal cavity immediately prior to assessment. To minimize data variables due to ambient light and temperature, the LDPI index was represented as the ratio of tumor pixels to scrotal pixels.

### Statistical Analysis

All data were represented as means ±SEM, and data were analyzed by one-way ANOVA with Fisher's adjustment. For survival analysis, survival rate represented by limb salvage score (Masaki I et al., FASEB J. 15: 1294-1296 (2001)) was analyzed using Kaplan-Mayer's method. The statistical significance of the survival experiments was determined using log-rank tests, and P<0.05 was considered to be statistically significant.

### Example 1

This Example shows that FGF-2 and PDGF-AA cooperatively enhance the expression of VEGF and HGF/SF via FGF-2-mediated upregulation of PDGFRα.

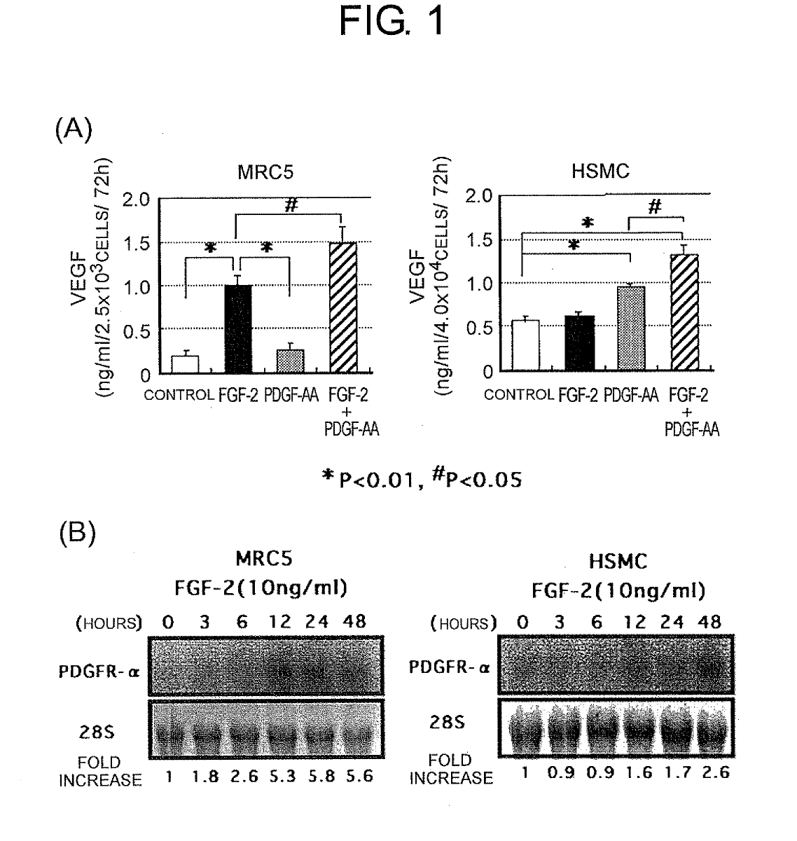

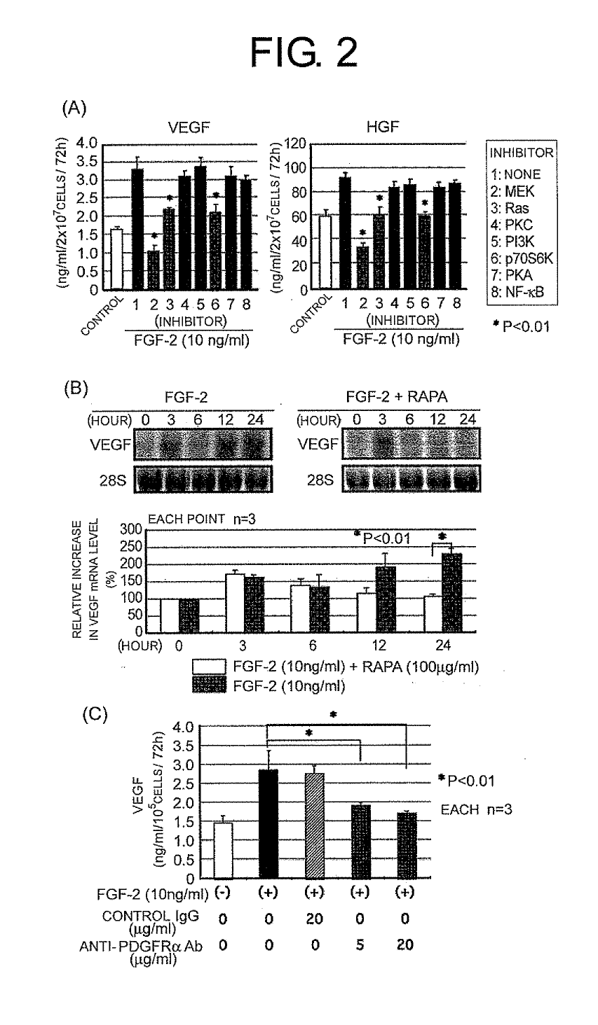

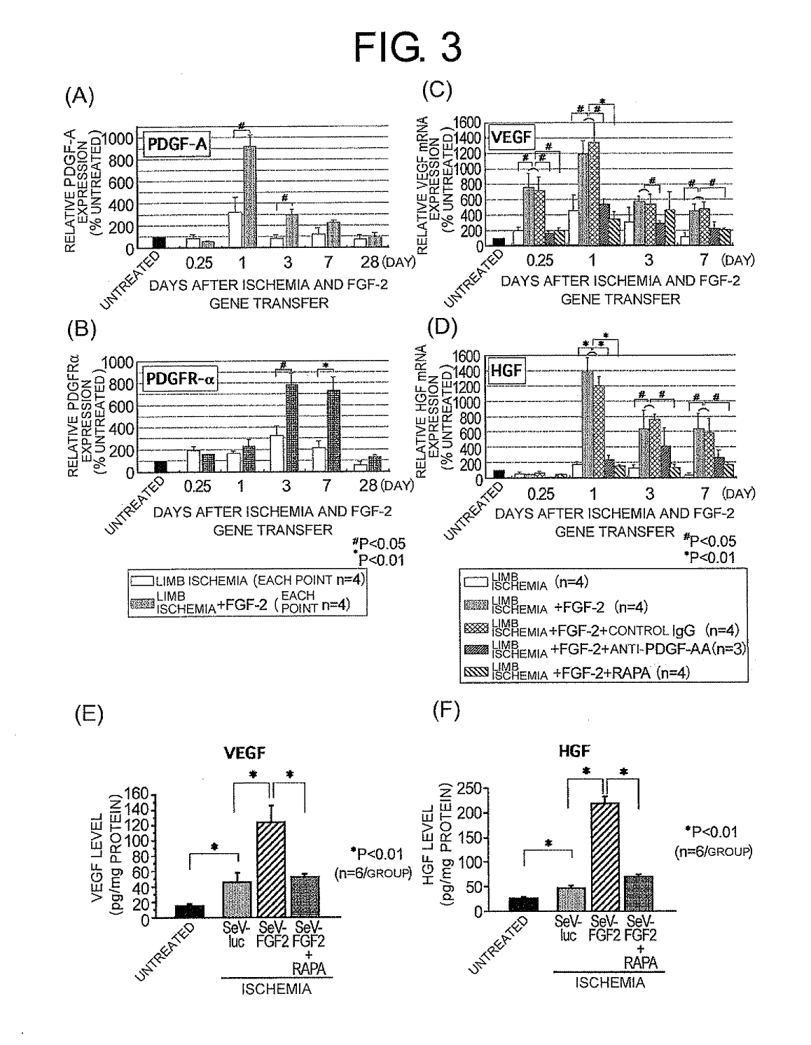

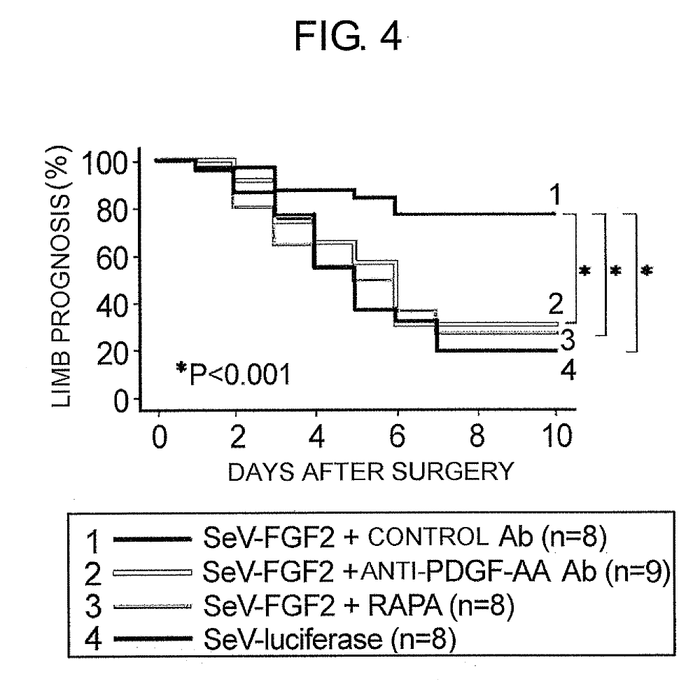

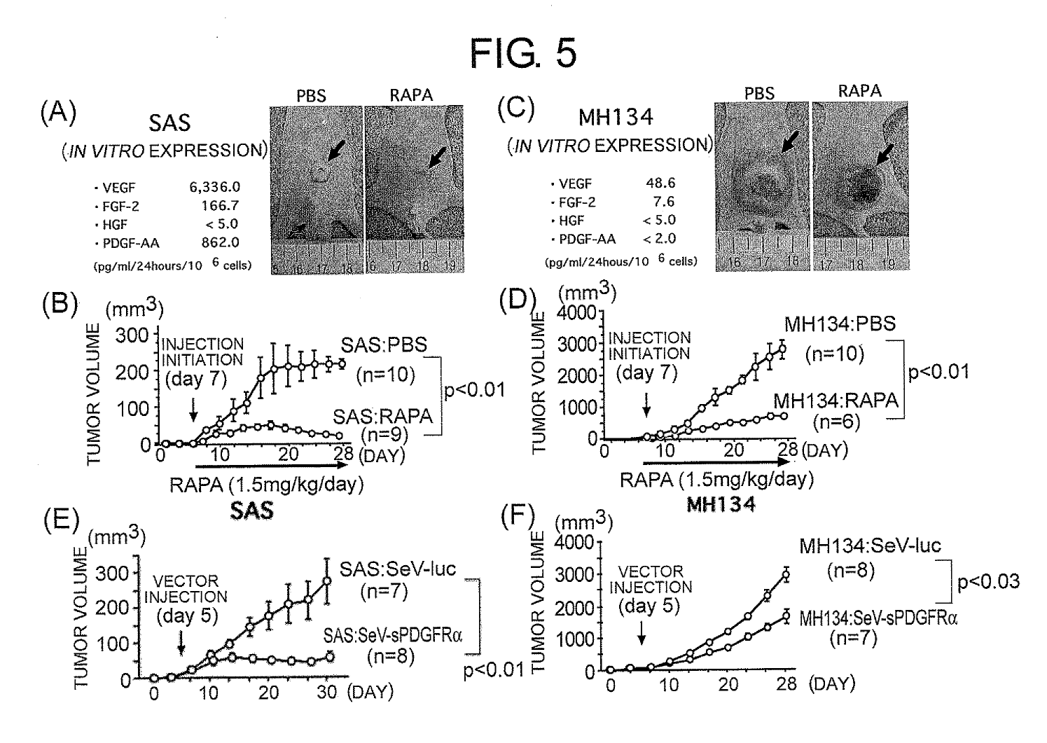

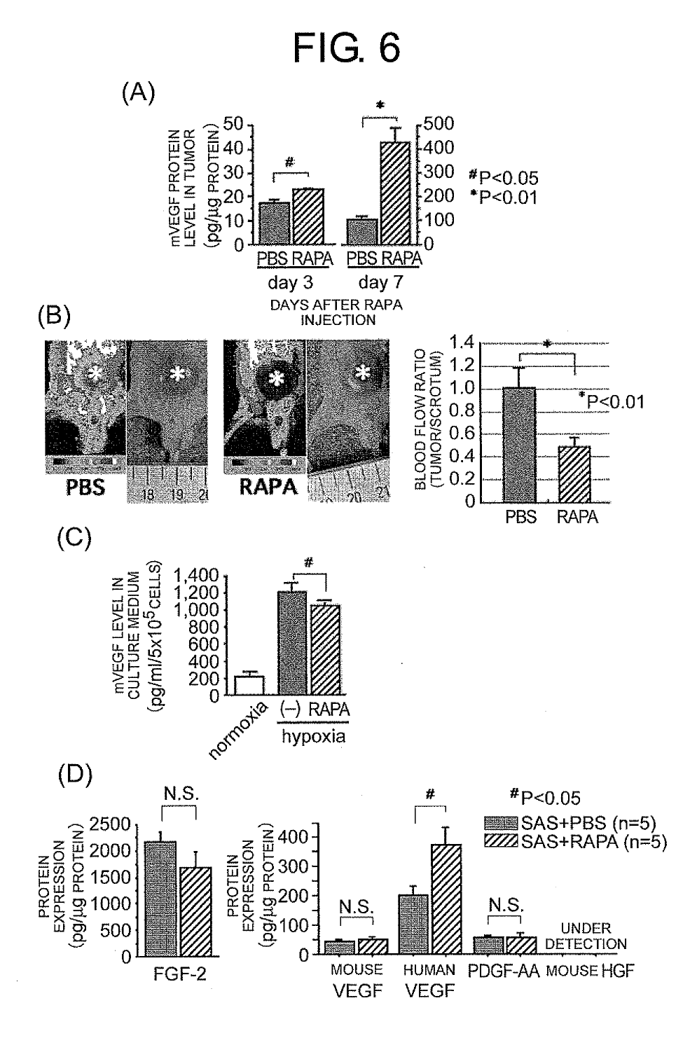

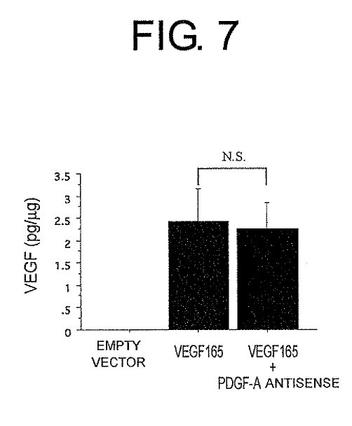

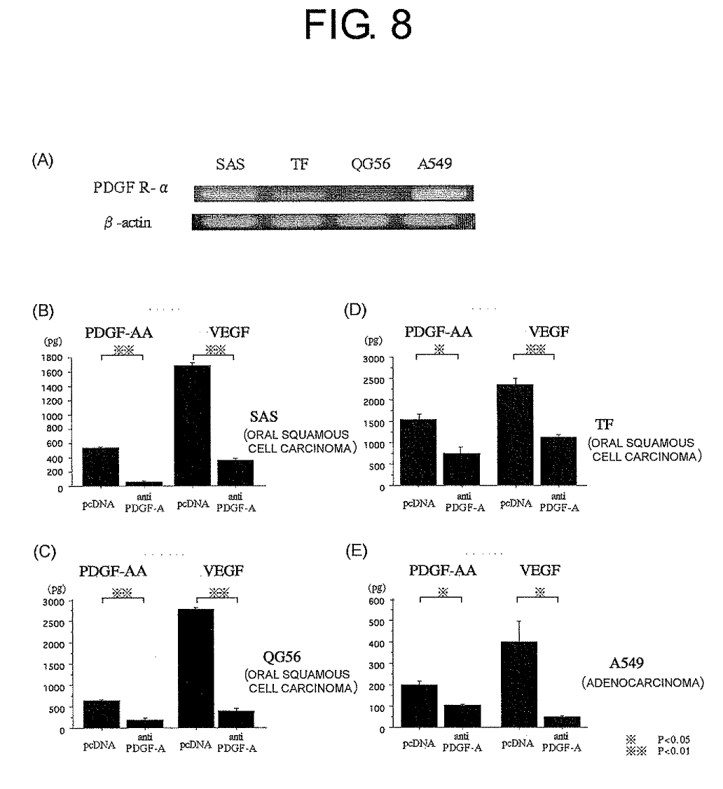

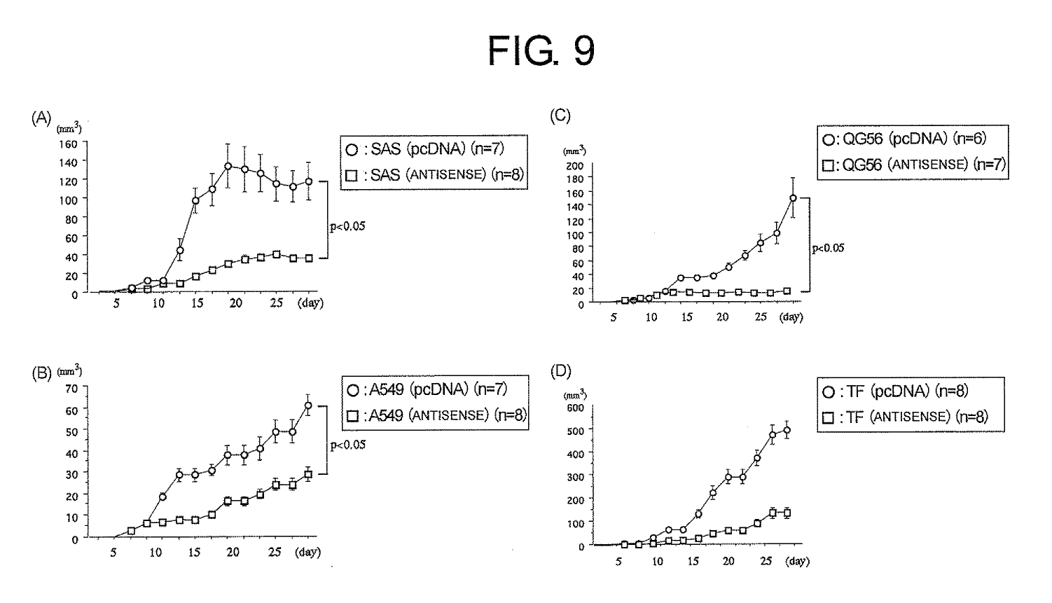

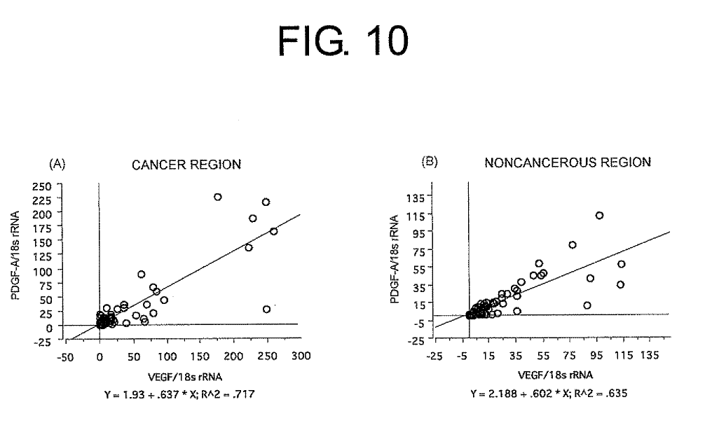

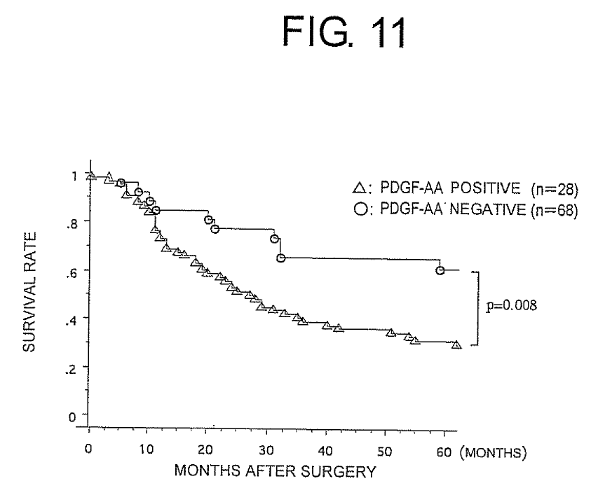

To assess the role of the PDGF-AA signal in the angiogenic response of host vasculature, the FGF-2-mediated induction of VEGF and HGF in human mesenchymal cells (MRC5 and HSMC) cultured under serum-free conditions was investigated. As shown in FIG. 1A, while FGF-2 stimulated release of VEGF into the culture medium of MRC5 cells, PDGF-AA did not (FIG. 1A, left). Conversely it was found that while PDGF-AA upregulated the level of VEGF in the culture medium of HSMC, FGF-2 did not (FIG. 1A, right). On the other hand, co-stimulation using FGF-2 and PDGF-AA was found to cooperatively enhance the expression of VEGF (FIG. 1A) and HGF/SF (data not shown) in both MRC5 and HSMC cell types. Since FGF-2 and PDGF-AA were also seen to have a cooperative effect on the expression of VEGF and HGF in mouse fibroblast cell line NIH3T3 (data not shown), as for MRC5 cells, this effect was shown to be common to mesenchymal cells, regardless of animal species. In clinical application such as ischemia treatments, angiogenesis might also be induced more effectively by administering both FGF-2 and PDGF-AA, rather than either one alone. Northern blot analysis showed FGF-2-mediated upregulation of PDGFRα transcription (FIG. 1B) in both MRC5 and HSMC cell types, but PDGF-AA did not change FGFR1 expression (data not shown). These findings suggest that FGF-2 modulates the PDGF-AA response, which modulates the expression of VEGF and HGF/SF in mesenchymal cells, via transcriptional regulation of PDGFRα.

### Example 2

This Example shows that in mesenchymal cells FGF-2 dependent expression of VEGF and HGF/SF is mediated by PDGFRα, and shut down by inhibition of the PDGFRα-p70S6K signal transduction pathway.

In addition to the cooperative effect of FGF-2 and PDGF-AA on the expression of VEGF and HGF/SF in MCs, the present inventors had previously discovered that FGF-2 enhances endogenous expression of PDGF-AA via Ras and p70S6K signal transductions, which contribute to the sustained expression of HGF/SF in HSMC (Onimaru M et al., Circ Res. 91: 723-730 (2002)). The present inventors hypothesized that an analogous system involving VEGF and MGF/SF expression also exists in fibroblasts (MRC5 cells). As seen in previous studies, FGF-2 typically upregulated the VEGF and HGF/SF proteins; and a MEK inhibitor, Ras-inhibitory peptide, and p70S6K inhibitor (RAPA) removed these effects (FIG. 2A). The repeated Northern blot analysis of time courses of FGF-2-mediated VEGF expression in MRC5 cells showed that biphasic (at three hours and after that) upregulation of VEGF occurs (FIG. 2B), as seen previously in HGF/SF expression using HSMC (Onimaru M et al., Circ Res. 91: 723-730 (2002)). Early phase VEGF expression was not affected by RAPA treatment, but RAPA treatment caused sustained expression in later phases to completely disappear (FIG. 2B). Moreover, FGF-2-mediated upregulation of VEGF protein was completely eliminated by an anti-PDGFRα antibody (FIG. 2C), as observed in RAPA treatment (FIG. 2A). Since the same result was obtained for HGF/SF expression (data not shown), it was concluded that the PDGFRα system plays a critical role in enhancing and sustaining FGF-2-mediated expression of VEGF and HGF/SF in MCs.

### Example 3

This Example shows that PDGFRα plays a critical role in the therapeutic effect of FGF-2 on mouse severe limb ischemia.

In order to investigate the predictable cascade-like relationship of FGF-2, PDGFRα and VEGF/HGF in vivo, two separate mouse limb ischemia models, namely, C57BL/6 mouse limb salvage model and balb/c nu/nu mouse limb autoamptation model (Masaki I et al., Circ Res. 90: 966-973 (2002)) were assessed in vivo using a recombinant Sendai virus (SeV-FGF2) that expresses FGF-2 (Masaki I et al., Circ Res. 90: 966-973 (2002); Onimaru M et al., Circ Res. 91: 723-730 (2002); Compagni A et al., Cancer Res. 60: 7163-7169 (2000); Yonemitsu Y et al., Nat. Biotechnol. 18: 970-973 (2000); Masaki I et al., FASEB J. 15: 1294-1296 (2001); Yamashita A et al., J. Immunol. 168: 450-457 (2002); Shoji F et al., Gene Ther. 10: 213-218 (2003)). FGF-2 overexpression was confirmed in the limb salvage model using ELISA assays (data not shown); however, upregulation of both PDGF-A and PDGFRα mRNA was confirmed by real-time quantitative PCR assays (FIGS. 3A and 3B). In the same tissue samples, expression of VEGF and HGF/SF were similarly enhanced by FGF-2, and an anti-PDGF-AA neutralizing antibody eliminated this effect, as did RAPS treatment (FIGS. 3C and 3D). The effect of RAPA was also confirmed at the protein level (FIGS. 3E and 3F). Moreover, since the anti-PDGF-AA antibody and RAPA eliminated the therapeutic effect of FGF-2 in the limb autoamptation model (FIG. 4), the PDGFRα system was shown to also play a critical role in FGF-2-mediated therapeutic angiogenesis.

### Example 4

This Example shows that inhibition of the PDGFRα-p70S6K signal transduction pathway induces tumor dormancy regardless of the diversity in expression of the angiogenic factors in each tumor type.

The results obtained using tumor-free systems suggest that the PDGFRα-p70S6K signal transduction pathway is essential for angiogenesis in MCs, and that RAPA mimics the effects of an anti-PDGF-AA antibody on FGF-2-mediated angiogenesis. However, there was some doubt as to whether RAPA could act regardless of angiogenetic stimulationin ubiquitous angiogenic reactions. To clarify this, two separate tumor cell lines were used to examine tumor angiogenesis. As the tumor cell lines, SAS, a cell line of human oral squamous cell carcinoma which expresses a high level of VEGF, FGF-2, and PDGF-AA; and MH134, a cell line of mouse hepatocarcinoma which secretes a much lower level of VEGF and FGF-2 than SAS, where no detectable expression of PDGF-AA is observed, were used.

As shown in FIGS. 5A to 5D, RAPA suppressed proliferation of both SAS and MH134 tumor types, suggesting that RAPA's antitumor effect is independent of the expression patterns of the angiogenic growth factors in each tumor type. To obtain further evidence showing that antitumor effects based on the PDGFRα-p70S6K pathway are independent of tumor type, SeV-hsPDGFRα that expresses a soluble form of human PDGFRα was injected into tumors, which were then assayed for tumor proliferation. As expected, SeV-hsPDGFRα significantly inhibited the proliferation of both tumors (FIGS. 5E and 5F). When tumor weights were measured at the termination of the experiment, the weights of tumors that received SeV-hsPDGFRα were significantly reduced in both tumor types compared with the control tumors that received the SeV vector expressing luciferase (SAS-luciferase; 415.1±104.4 mg vs. SAS-hsPDGFRα: 54.3±9.6 mg, MH134-luciferase: 3,930.4±304.4 mg vs. MH134-hsPDGFRα: 2,654.4±296.5 mg; P=0.0027 and P=0.0106, respectively, mean ±S.E.).

Considering that RAPA treatment has antitumor effects other than those based on p70S6K inhibition, such as direct inhibition of endothelium proliferation (Vinals F et al., J Biol. Chem. 274: 26776-26782 (1999); Yu Y et al., J Cell Physiol. 178: 235-246 (1999)), the inhibitory effects of SeV-hsPDGFRα on the PDGFRα-p70S6K pathway are very high, indicating that tumor proliferation can be suppressed more efficiently using multiple administrations.

To confirm that antitumor effects caused by inhibition of the PDGFRα-p70S6K signal transduction pathway are independent of the expression patterns of angiogenic factors, the in vivo and in vitro expression of VEGF in the presence or absence of RAPA was examined. In culture systems, 100 ng/ml of RAPA significantly reduced the endogenous secretion of VEGF in SAS to about 30% to 50% of basal levels. Similar reductions were seen in other examined tumors (oral squamous cell carcinomas: QG56, TF, KN, and EBC-1, and adenocarcinoma: PC9) under conditions of normoxia. Similar findings were reported by other groups (Guba M et al., Nat. Med. 8:128-135 (2002)). The effect of RAPA on the expression of PDGF-AA and FGF-2 in each tumor type was not observed (data not shown). However, in the in vivo evaluation of MH134 tumors, VEGF expression was significantly increased three or seven days after RAPA treatment, compared with a buffer-treated control (FIG. 6A). Furthermore, Doppler perfusion image analysis revealed that blood flow in both tumors was reduced seven days after beginning RAPA injections (FIG. 6B).

These results can be explained as follows: RAPA treatment induces hypoxia, which results in upregulation of VEGF via a hypoxia-dependent mechanism, thereby counteracting the RAPA-mediated downregation. This mechanism was confirmed as follows: In MH134 cultures, RAPA shows a significant but only minimal effect on hypoxia (2.5% O₂)-induced VEGF expression (FIG. 6C). Similar results were obtained in all cell lines examined (data not shown).

Accordingly, an SAS xenograft model was employed to examine origin of VEGF using human- or mouse-specific ELISA systems. RAPA significantly increased human VEGF levels without affecting murine VEGF levels (FIG. 6D), showing that the increase in tumor cell-derived VEGF levels was mediated by hypoxia due to angiogenesis targeting at the host vasculature, regardless of the diversity of angiogenic factor expression in each tumor type.

### Example 5

This Example exemplifies suppression of tumor proliferation by inhibiting PDGF-A expression.

Cloning of human PDGF-A gene was carried out as follows: Using cDNAs prepared by reverse transcription of mRNAs from MRC5 cells (Isogen, Oligo dT primers were used), PCR was carried out using the forward primer AAGAATTCATGAGGACCTTGGCTTGCCTGC (SEQ ID NO: 26) and the reverse primer AAGAATTCTTAGGTGGGTTTTAACCTTTTTCTTTT (SEQ ID NO: 27) (Underlines indicate EcoRI sites). After five minutes at 96° C., 35 cycles of 30 seconds at 96° C., 45 seconds at 60° C. and 45 seconds at 72° C. were carried out, followed by five minutes at 72° C. The PCR product (636 bp) was subcloned into TA cloning vector pCR II (registered trademark, Invitrogen). After confirming the nucleotide sequence by sequencing, the product was cut out using a restriction enzyme EcoRI, then subcloned into the expression vector pcDNA 3.1 (+) (registered trademark, Invitrogen). The product was cleaved with a restriction enzyme SacI to confirm its orientation, and the antisense gene was identified (pcDNA3-asPDGFA).

In order to examine the effect of presence or absence of PDGF-A expression on the expression of the exogenously introduced VEGF gene, the human VEGF165-expressing plasmid vector (pcDNA3-hVEGF165) and the antisense human PDGF-A-expressing vector (pcDNA3-asPDGFA) were simultaneously introduced into NIH3T3 cells. To prepare control cells, an empty vector pcDNA 3.1) or human VEGF165-expressing plasmid vector (pcDNA3-hVEGF165) alone was introduced into cells, and the VEGF expression Levels were compared. As a result, VEGF expression was undetectable in cells introduced with the empty vector (pcDNA 3.1), and the VEGF expression level in the cells introduced with pcDNA3-hVEGF165 alone was 2.42±0.73 (mean ±S.E.) pg/μg protein, but 2.27±0.57 pg/μg protein in the cells co-introduced with pcDNA3-hVEGF165 and pcDNA3-asPDGFA, indicating that the VEGF165 level is not significantly affected by the introduction or otherwise of pcDNA3-asPDGFA, namely, antisense PDGF-A does not interfere with exogenous VEGF expression (FIG. 7).

The antisense human PDGF-A expression vector (pcDNA3-asPDGFA) was introduced into human squamous carcinomas or adenocarcinomas to generate stable transformed cell lines. Specifically, pcDNA3-asPDGFA was transfected into tumor cell lines (SAS, TF, QG56, and A549) using Lipofectamine (registered trademark, Life Technologies), followed by culture in the presence of 500 μg/ml of G418 (Promega) to obtain the transformed tumor cell lines. These cells were used for single colony culture in a 96 well plate, then ELISA was used to select colonies where PDGF-A expression is strongly suppressed. This process was repeated three times. 5×10⁵ of the tumor cells thus obtained were plated on a 6 well plate, cultured overnight washed twice with a serum-free RPMI 1640 medium, and then incubated in 1 ml of the same medium for 24 hours. Subsequently, cells were harvested and the expression levels of PDGF-AA were quantitatively determined using PDGF-AA ELISA kits (R&D). The levels of VEGF secreted into the culture medium were similarly quantified by ELISA. Tumor cells introduced with an empty vector were generated as controls.

FIG. 8 (A) shows the results of using RT-PCR to determine the expression level of PDGFRα in each cancer cell type. All of the target tumors were found to express PDGFRα. When antisense human PDGF-A expression vector was introduced into these tumor cells, not only was the expression level of PDGF-AA significantly reduced in all of the tumor cells, but the expression level of VEGF was also decreased (FIGS. 8(B) to (E)).

Tumor implant assays were then used to examine changes in the tumor proliferative ability of the tumor cells in which PDGF-A expression was inhibited. 1×10⁶ of the above produced transformed tumor cells were subcutaneously injected into lateral region of Balb/c nude mice (5 weeks old, male). After that, tumor size was measured three times a week. Tumor volume was calculated by π/6*a*b*c (a, b, and c are transverse diameter, longitudinal diameter, and width, respectively). As shown in FIG. 9, a clear decrease in the tumor proliferation was found in all of the tumor cells expressing antisense PDGF-A. In addition, there was no significant difference in the in vitro proliferative ability of these cells.

Real-time PCR was used to examine the correlation between the mRNA expression of PDGF-A and VEGF in fresh surgical specimens from human lung cancers. Specifically, cDNAs were prepared by reverse transcription of mRNAs from human lung cancer tissues or normal tissues, followed by purification (Isogen, Oligo dT primer were used), and these were used to quantitatively determine PDGF-A mRNAs by real-time PCR using ABI 7000. The nucleotide sequences of the forward primer, reverse primer, and Taqman probe (FAM, TAMRA) for real-time PCR were TCCACGCCACTAAGCATGTG (SEQ ID NO: 28), TCGACCTGACTCCGAGGAAT (SEQ ID NO: 29), and CTGCAAGACCAGGACGGTCATTTACGA (SEQ ID NO: 30), respectively. Conditions for PCR were two minutes at 52° C., followed by ten minutes at 96° C., and 40 cycles of 15 seconds at 95° C. and one minute at 60° C. As a result expression of PDGF-A and VEGF were found to have a significant correlation in both cancer and noncancerous regions (FIG. 10). These results suggest that systems for inducing VEGF expression via the autocrine action of PDGF-A have been established not only for normal tissues but also for cancers.

The correlation between the PDGF-AA positive rate and patient prognosis was also examined using surgical specimens from human lung cancers. To examine PDGF-AA expression in the surgical specimens from human lung cancers by immunohistochemical staining, tissue sections of the human lung cancer tissues were deparaffinized and washed three times with PBS. After blocking the sections with 3% skimmed milk for 30 minutes, they were reacted overnight at 4° C. with the primary antibody (anti-human PDGF-AA antibody, 60-fold diluted, R&D). After washing three times with PBS, they were reacted with the secondary antibody (Histofine Simple Stein MAX PO (G), Nichirei Corp.) at room temperature for 30 minutes, followed by color development using DAB. As shown in FIG. 11, the prognosis of PDGF-AA-positive lung cancer patients was significantly lower than that of PDGF-AA-negative patients. From these results, it is possible to predict tumor malignancy and patient prognosis by testing the expression level of PDGF-A. Namely, if PDGF-A expression is detected by determining PDGF-A expression in a tumor, the tumor is considered to be malignant compared with PDGF-A expression-negative tumors, which indicates a poor prognosis. Moreover, the results show that inhibition of PDGF-A expression and/or activity is effective in antitumor therapies against PDGF-A-positive cancers.

### INDUSTRIAL APPLICABILITY

The present invention provides methods for suppressing tumor proliferation by inhibiting the expression of PDGF-A or the binding between PDGF-A homodimers and PDGFRα. Activation of the PdGFRα-p70S6K signal transduction pathway by PDGF-AA is an important factor in tumor angiogenesis and related to the prognosis of patients suffering from tumors. By inhibiting PDGF-A expression in tumors or surrounding tissues, or by inhibiting the binding between PDDGF-A homodimers and PDGFRα, it is possible to inhibit tumor angiogenesis, thereby suppressing tumor proliferation.

1. A method for suppressing tumor proliferation, comprising the step of inhibiting the expression of a PDGF-A or the binding between a PDGF-A homodimer and a PDGFRα.

2. The method of claim 1, wherein the step administers to a tumor a minus strand RNA virus vector encoding a secretory protein that binds to a PDGF-A homodimer or a PDGFRα.

3. The method of claim 2, wherein a cell to which the vector has been introduced is administered.

4. The method of claim 3, wherein the cell is a dendritic cell.

5. The method of claim 2, wherein the secretory protein is a soluble PDGFRα.

6. The method of claim 2, wherein the minus strand RNA virus vector is a Sendai virus vector.

7. The method of claim 1, wherein the step administers to a tumor an antisense RNA or siRNA of a PDGF-A gene, or a vector encoding the antisense RNA or siRNA.

8. The method of claim 1, wherein the tumor is selected from the group consisting of a squamous cell carcinoma, a hepatocarcinoma, and an adenocarcinoma.

9. An antitumor agent comprising a compound that inhibits the expression of a PDGF-A or the binding between a PDGF-A homodimer and a PDGFRα as an active ingredient.

10. The antitumor agent of claim 9, wherein the agent comprises any one of (a) to (d) below:
(a) a secretory protein that binds to a PDGF-A homodimer or a PDGFRα,
(b) an antisense RNA of a PDGF-A gene or a PDGFRα gene,
(c) an siRNA of a PDGF-A gene or a PDGFRα gene, and
(d) a vector encoding any one of (a) to (c).

11. The antitumor agent of claim 10, wherein the agent comprises a minus strand RNA virus vector encoding a secretory protein that binds to a PDGF-A homodimer or a PDGFRα.

12. The antitumor agent of claim 10 or 11, wherein the secretory protein is a soluble PDGFRα.

13. The antitumor agent of claim 11, wherein the minus strand RNA virus vector is a Sendai virus vector.

14. The antitumor agent of claim 10, wherein the agent comprises a cell, to which has been introduced a vector that encodes a secretory protein that binds to a PDGF-A homodimer or a PDGFRα.

15. The antitumor agent of claim 14, wherein the cell is a dendritic cell.

16. The antitumor agent of claim 10, wherein the agent comprises an antisense RNA or siRNA of a PDGF-A gene, or a vector encoding the antisense RNA or siRNA, as an active ingredient.

17. The antitumor agent of claim 9, wherein the tumor is selected from the group consisting of a squamous cell carcinoma, a hepatocarcinoma, and an adenocarcinoma.


SEQUENCE LISTING (30 sequences)

SEQ ID NO: 1 - DNA, 2797 bp, Homo sapiens
  CDS (839)..(1471)
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgttcaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 2 - protein, 211 aa, Homo sapiens [sequence omitted]

SEQ ID NO: 3 - DNA, 2740 bp, Homo sapiens
  CDS (839)..(1426)
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgttcaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 4 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 5 - DNA, 6633 bp, Homo sapiens
  CDS (395)..(3661)
ttctccccgccccccagttgttgtcgaagtctgggggttgggactggaccccctgattgcgtaagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacaggaggagactgcaagagatcatttgggaaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccggatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctctcgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttcttcatgcctgatgaaagctttggcaaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgggtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtt

SEQ ID NO: 6 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 7 - DNA, 1596 bp, Artificial
  a human soluble PDGFR-alpha cDNA
atggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaagactacaaggacgacgatgacaag

SEQ ID NO: 8 - protein, 532 aa, Artificial
  Synthetic Construct
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSEDYKDDDDK

SEQ ID NO: 9 - DNA, 28 bp, Artificial
  an artificially synthesized sequence
aaagatctatggggacttcccatccggc

SEQ ID NO: 10 - DNA, 50 bp, Artificial
  an artificially synthesized sequence
ttgctagctcacttgtcatcgtcgtccttgtagtcttcagaacgcagggt

SEQ ID NO: 11 - DNA, 21 bp, Artificial
  an artificially synthesized sequence
gcaggctgctgtaacgatgaa

SEQ ID NO: 12 - DNA, 22 bp, Artificial
  an artificially synthesized sequence
tcacatctgctgtgctgtagga

SEQ ID NO: 13 - DNA, 26 bp, Artificial
  an artificially synthesized sequence
catgcagatcatgcggatcaaacctc

SEQ ID NO: 14 - DNA, 24 bp, Artificial
  an artificially synthesized sequence
cagcaataccatttggaatggaat

SEQ ID NO: 15 - DNA, 24 bp, Artificial
  an artificially synthesized sequence
ttgaagttctcgggagtgatatca

SEQ ID NO: 16 - DNA, 25 bp, Artificial
  an artificially synthesized sequence
cgttgggattcgcagtaccctcaca

SEQ ID NO: 17 - DNA, 19 bp, Artificial
  an artificially synthesized sequence
cgtcaagtgccagccttca

SEQ ID NO: 18 - DNA, 21 bp, Artificial
  an artificially synthesized sequence
atgcacactccaggtgttcct

SEQ ID NO: 19 - DNA, 24 bp, Artificial
  an artificially synthesized sequence
cactttggccaccttgacactgcg

SEQ ID NO: 20 - DNA, 24 bp, Artificial
  an artificially synthesized sequence
gagcatcttcgacaacctctacac

SEQ ID NO: 21 - DNA, 25 bp, Artificial
  an artificially synthesized sequence
ccggtatccactcttgatcttattg

SEQ ID NO: 22 - DNA, 27 bp, Artificial
  an artificially synthesized sequence
ccctatcctggcatgatggtcgattct

SEQ ID NO: 23 - DNA, 22 bp, Artificial
  an artificially synthesized sequence
cctggagaaacctgccaagtat

SEQ ID NO: 24 - DNA, 22 bp, Artificial
  an artificially synthesized sequence
ttgaagtcgcaggagacaacct

SEQ ID NO: 25 - DNA, 26 bp, Artificial
  an artificially synthesized sequence
tgcctgcttcaccaccttcttgatgt

SEQ ID NO: 26 - DNA, 30 bp, Artificial
  an artificially synthesized sequence
aagaattcatgaggaccttggcttgcctgc

SEQ ID NO: 27 - DNA, 35 bp, Artificial
  an artificially synthesized sequence
aagaattcttaggtgggttttaacctttttctttt

SEQ ID NO: 28 - DNA, 20 bp, Artificial
  an artificially synthesized sequence
tccacgccactaagcatgtg

SEQ ID NO: 29 - DNA, 20 bp, Artificial
  an artificially synthesized sequence
tcgacctgactccgaggaat

SEQ ID NO: 30 - DNA, 27 bp, Artificial
  an artificially synthesized sequence
ctgcaagaccaggacggtcatttacga